(19) United States
(12) Patent Application Publication
(10) Pub. No.: US 2021/0245157 A1
(43) Pub. Date: Aug. 12, 2021

(54) PORTABLE MICROFLUDIC SYSTEM FOR BIOLOGICAL AND ANALYTICAL TESTING

(71) Applicants: MicrobeDX, Inc., Pacific Palisades, CA (US); The Regents of the University of California, Oakland, CA (US)

(72) Inventors: Marc Madou, Irvine, CA (US); Alexandra Perebikovsky, Irvine, CA (US); Yujia Liu, Irvine, CA (US); Horacio Kido, Irvine, CA (US); Scott Adam Churchman, Santa Monica, CA (US); Colin Wynn Halford, Los Angeles, CA (US)

(21) Appl. No.: 17/054,306

(22) PCT Filed: May 14, 2019
(86) PCT No.: PCT/US2019/032220

§ 371 Date: Nov. 10, 2020

Related U.S. Application Data
(60) Provisional application No. 62/671,392, filed on May 14, 2018.
(60) Provisional application No. 62/801,395, filed on Feb. 5, 2019.

(51) Int. Cl. B01L 3/00; G01N 21/64

(52) U.S. Cl. CPC B01L 3/502738; G01N 21/6458; C12Q 1/18; B01L 2400/0409; B01L 2300/048; B01L 2300/0803

(57) ABSTRACT

Disclosed herein are an apparatus, system, and methods related to a portable microfluidic system for biological and analytical testing of biological fluids. In particular, the system comprises the use of a rotating microfluidic platform technology to manipulate and perform sample-to-answer assays on biological fluids. More particularly, the system described herein may be capable of performing bacteria identification/quantification (IDQ) and/or antimicrobial susceptibility testing (AST).

### CROSS REFERENCE TO RELATED APPLICATION

The present application claims the benefit under 35 U.S.C. § 119(e) of provisional patent application Ser. No. 62/671,392, filed on May 14, 2018, entitled SYSTEMS AND DEVICES FOR MICROBIAL DETECTION, and provisional patent application Ser. No. 62/801,395, filed on Feb. 5, 2019, entitled PORTABLE MICROFLUIDIC SYSTEM FOR BIOLOGICAL AND ANALYTICAL TESTING, the entirety of which are hereby incorporated by reference.

### FIELD OF THE INVENTION

The subject disclosure relates to a portable microfluidic system and associated methods for biological and analytical testing of biological fluids. More particularly, the subject disclosure relates to a rotating cartridge system that is capable of manipulating and analyzing biological fluids.

### BACKGROUND

The analysis of biological fluid samples, particularly the detection of certain target molecules within a biological fluid, has many clinical applications. For example, the isolation and identification of uropathogens in urine samples is an important aspect of the clinical management of patients with urinary tract infections (UTIs) and other infectious diseases, such as bacteremia in patients with sepsis.

Culture-based methods for isolating and identifying uropathogens are known in the art; however, these methods can be time consuming, labor intensive, and are not cost effective. Recent advances in technology have allowed for the development of electrochemical DNA biosensors with molecular diagnostic capabilities, including bacterial pathogen detection. To run a successful electrochemical assay, a target cell can first be lysed such that a nucleic acid molecule, such as RNA, can be released from within the cell. Thus, the use of electrochemical DNA biosensors relies on the efficient lysis and release of target molecules from the cells to be diagnosed. These cells may include, among others, prokaryotic cells such as Gram-negative bacteria or Gram-positive bacteria, or fungal cells, such as yeast.

In some circumstances, a biological fluid may contain microorganisms, such as bacteria, and it may be desirable to determine if a given microorganism is susceptible to treatment by one or more antimicrobial agents. For example, if a biological fluid contains bacteria, it may be useful to determine if the particular bacteria in the sample is susceptible to, or alternatively, is resistant to, one or more antibiotics. The effectiveness of an antibiotic can vary with the resistance of a bacterial pathogen to the antibiotic. Therefore, determining the antimicrobial sensitivity of bacterial pathogens in a clinical specimen is a key step in the diagnosis and treatment of infectious diseases.

Two common methods of phenotypic antimicrobial susceptibility testing (“AST”) are broth microdilution and Kirby-Bauer disc diffusion. While such methods can be relatively accurate in determining the antimicrobial sensitivity of bacterial pathogens in clinical specimen, both are relatively slow, requiring lengthy incubation times of the sample with the antibiotics (up to 24 hours). Such methods also often require a lengthy pre-incubation culturing period (24-72 hours) to generate the AST sample, can be relatively labor-intensive, and can be challenging to automate.

Due to the relatively serious nature of infectious diseases, it can be the case that treatment should not be delayed. Therefore, antibiotic treatment is frequently started before AST results can be obtained using conventional, non-molecular, and slow-acting testing methods. This can lead to a patient being given antibiotics, or other antimicrobial agents, without first knowing if the particular bacteria afflicting the patient is susceptible or resistant to the particular antibiotic administered. If the bacteria are in fact resistant, the initial course of antibiotics may be ineffective, which may contribute to a known problem/trend of patients receiving unnecessary or less effective antibiotics when other, potentially more effective antibiotics may have been available for use. With the rise in antibiotic-resistant bacteria, there is an urgent need to develop methods for rapidly determining the antibiotic susceptibility of certain bacteria.

Despite the advances made to date in identifying bacteria and determining the antimicrobial sensitivity of bacterial pathogens in a clinical specimen, there is room for improvement to address the abovementioned problems and shortcomings of the prior art.

### SUMMARY

It is an object of the present invention to obviate or mitigate at least one of the abovementioned disadvantages of the prior art.

It is another object of the present invention to provide a novel portable system for biological and analytical testing of biological fluids.

Accordingly, in one of its aspects, the present invention provides a rotatable microfluidic cartridge for processing and analyzing at least one clinical specimen, comprising: a body portion having a first surface and an opposing second surface; a fluid inlet region configured to receive the clinical specimen; and at least one microfluidic flow path connected to the fluid inlet region and extending downstream from the fluid inlet region through the body of the cartridge, wherein the cartridge is configured to be secured with respect to a driving apparatus and configured to be rotated about a rotational axis when the driving apparatus is in operation.

In another of its aspects, the present invention provides a rotatable microfluidic cartridge for processing and analyzing at least one clinical specimen, comprising: a body portion having a first surface and an opposing second surface; a fluid inlet region configured to receive the clinical specimen; and at least a first microfluidic flow path extending downstream from the fluid inlet port through the body of the cartridge, wherein the cartridge is configured to be secured with respect to a driving apparatus and configured to be rotated about a rotational axis when the driving apparatus is in operation, and wherein the first microfluidic flow path comprises two or more fluid conduits connected to one another through a series of microfluidic flow channels that are configured to allow the clinical specimen to flow between the fluid conduits when the cartridge is rotated or oscillated.

In another of its aspects the present invention provides a rotatable microfluidic cartridge for processing and analyzing a clinical specimen comprising a body portion configured to be secured with respect to a driving apparatus and configured to be rotated about a rotational axis, a fluid inlet region configured to receive the clinical specimen, and at least a first microfluidic flow path extending downstream from the fluid inlet port, wherein the microfluidic flow path comprises: (a) an incubation chamber; (b) a lysis chamber; (c) a neutralization region; (d) a detection/hybridization chamber; (e) at least one waste chamber; (f) at least one wash chamber comprising a wash buffer; wherein (a), (b), (c), (d), (e) and (f) are all connected through a series of microfluidic channels that are configured to allow the clinical specimen to flow from (a) to (b) to (c) to (d) to (e) and to allow the wash buffer to flow from (f) to (d) when the cartridge is rotated.

In another of its aspects, the present invention provides a rotatable microfluidic cartridge for processing and analyzing a clinical specimen, comprising: a body portion having a and an opposing second surface and configured to be secured with respect to a driving apparatus and configured to be rotated about a rotational axis; a fluid inlet region configured to receive the clinical specimen; and at least a first microfluidic flow path extending downstream from the fluid inlet port, wherein the microfluidic flow path comprises:
(a) an incubation chamber located downstream from the fluid inlet region, wherein the incubation chamber is configured to receive the clinical specimen from the fluid inlet region when the cartridge is rotated, and wherein the incubation chamber is configured to allow turbulent mixing within the chamber when the cartridge oscillated back and forth along an oscillation path at a predetermined oscillation frequency;
(b) a lysis chamber connected to and located downstream from the incubation chamber, wherein the lysis chamber is configured to receive the clinical specimen from the incubation chamber when the cartridge is rotated, wherein the lysis chamber comprises at least one magnetic lysis puck that can translate within the chamber to cause disruption of a cellular membrane in the clinical specimen when the cartridge is rotated, and wherein the lysis chamber contains at least one chemical lysing agent;
(c) a neutralization region connected to and located downstream from the lysis chamber, wherein the neutralization region is configured to receive the clinical specimen from the lysis chamber when the cartridge is rotated, and wherein the neutralization region comprises a buffer solution capable of neutralizing the clinical specimen and a series of detector probes to hybridize and bind with target molecules in the sample, wherein the detector probes are functionalized with a fluorescent signaling molecule; and
(d) a detection/hybridization chamber connected to and located downstream from the neutralization region, wherein the detection/hybridization chamber is configured to receive the clinical specimen from the neutralization region when the cartridge is rotated, and wherein the detection/hybridization chamber comprises a series of capture probes, wherein the capture probes are immobilized on a solid surface (e.g., plastic) surface such that the neutralized clinical specimen is allowed to hybridize and bind with the capture probes when the microfluidic cartridge is oscillated back and forth along an oscillation path at a predetermined oscillation frequency.

In another of its aspects, the present invention provides a rotatable microfluidic cartridge for processing and analyzing at least one clinical specimen comprising a body portion having a and an opposing second surface, a fluid inlet region configured to receive the clinical specimen, and at least a first microfluidic flow path extending downstream from the fluid inlet port through the body of the cartridge, wherein the cartridge is configured to be secured with respect to a driving apparatus and configured to be rotated about a rotational axis when the driving apparatus is in operation, and wherein the first microfluidic flow path comprises:
(a) two or more fluid conduits connected to one another through a series of microfluidic flow channels that are configured to allow the clinical specimen to flow between the fluid conduits when the cartridge is rotated or oscillated; and
(b) a first blocking member positioned in the microfluidic flow channel between the first fluid conduit and the second fluid conduit and fluidly isolating the second fluid conduit from the first fluid conduit, the first blocking member being spaced inwardly from the first and second surface of the body;
wherein the first blocking member is configured to be at least partially destroyable via an actuator that is external to body portion of microfluidic cartridge when the cartridge is in use, thereby establishing fluid communication between the first fluid conduit and the second fluid conduit and allowing the clinical specimen to flow from the first fluid conduit to the second fluid conduit.

In another of its aspects, the present invention provides a rotatable microfluidic cartridge for processing and analyzing at least one clinical specimen comprising a body portion having a first surface and an opposing second surface; a fluid inlet region configured to receive the clinical specimen and at least a first microfluidic flow path extending downstream from the fluid inlet port through the body of the cartridge, wherein the cartridge is configured to be secured with respect to a driving apparatus and configured to be rotated about a rotational axis when the driving apparatus is in operation, and wherein the first microfluidic flow path comprises:
(a) a series of two or more fluid conduits connected to one another through a series of microfluidic flow channels that are configured to allow the clinical specimen to flow between the fluid conduits when the cartridge is rotated or oscillated; and
(b) a blocking member positioned in the microfluidic flow channel between each of the two or more fluid conduits fluidly isolating each of the fluid conduits from one another, each of the blocking members being spaced inwardly from the first surface and second surface of the body;
wherein each of the blocking members is configured to be at least partially destroyable via an actuator that is external to the body portion of the microfluidic cartridge when the cartridge is in use, thereby establishing fluid communication between the two or more fluid conduits at a predetermined time and allowing the clinical specimen to flow from the first fluid conduit to the second fluid conduit and so on.

In another of its aspects, the present invention provides a system for processing and analyzing a sample comprising:
(a) a driving apparatus, wherein the driving apparatus may be configured to receive a microfluidic cartridge and to rotate or oscillate the cartridge; and
(b) a rotatable microfluidic cartridge for processing and analyzing a clinical specimen comprising a body portion configured to be secured with respect to the driving apparatus and configured to be rotated about a rotational axis, a fluid inlet region configured to receive the clinical specimen, and at least a first microfluidic flow path extending downstream from the fluid inlet port.

In another of its aspects, the present invention provides a system for processing and analyzing a sample comprising:
(a) a driving apparatus comprising a rotatable motor, an array of permanent magnets, a laser and a fluorescent detection system, wherein the driving apparatus may be configured to receive a microfluidic cartridge and to rotate the cartridge; and
(b) a rotatable microfluidic cartridge for processing and analyzing a clinical specimen comprising a body portion configured to be secured with respect to the driving apparatus and configured to be rotated about a rotational axis, a fluid inlet region configured to receive the clinical specimen, and at least a first microfluidic flow path extending downstream from the fluid inlet port, wherein the microfluidic flow path comprises:
   i. an incubation chamber located downstream from the fluid inlet region, wherein the incubation chamber is configured to receive the clinical specimen from the fluid inlet region when the cartridge is rotated, and wherein the incubation chamber is configured to allow turbulent mixing within the chamber when the cartridge oscillated back and forth along an oscillation path at a predetermined oscillation frequency;
   ii. a lysis chamber connected to and located downstream from the incubation chamber, wherein the lysis chamber is configured to receive the clinical specimen from the incubation chamber when the cartridge is rotated, wherein the lysis chamber comprises at least one magnetic lysis puck that can translate within the chamber to cause disruption of a cellular membrane in the clinical specimen when the cartridge is rotated, and wherein the lysis chamber contains at least one chemical lysing agent;
   iii. a neutralization region connected to and located downstream from the lysis chamber, wherein the neutralization region is configured to receive the clinical specimen from the lysis chamber when the cartridge is rotated, and wherein the neutralization region comprises a buffer solution capable of neutralizing the clinical specimen and a series of detector probes to hybridize and bind with target molecules in the sample, wherein the detector probes are functionalized with a fluorescent signaling molecule; and
   iv. a detection/hybridization chamber connected to and located downstream from the neutralization region, wherein the detection/hybridization chamber is configured to receive the clinical specimen from the neutralization region when the cartridge is rotated, and wherein the detection/hybridization chamber comprises a series of capture probes, wherein the capture probes are immobilized on a solid surface (e.g., plastic) such that the neutralized clinical specimen is allowed to hybridize and bind with the capture probes when the microfluidic cartridge is oscillated back and forth along an oscillation path at a predetermined oscillation frequency.

In another of its aspects, the present invention provides a method of performing bacteria identification and quantification of a clinical specimen, using the system and devices disclosed herein, the method comprising the steps of:
(a) securing a microfluidic cartridge with respect to a driving apparatus with a rotating motor;
(b) dispensing a precise volume of the clinical specimen into the fluid inlet region of the microfluidic cartridge;
(c) using the rotating motor of the driving apparatus to rotate the microfluidic cartridge at a predetermined speed to allow the clinical specimen within the fluid inlet port to flow into the first fluid conduit;
(d) using the rotating motor of the driving apparatus to oscillate the microfluidic cartridge at a predetermined oscillation frequency and angle to allow turbulent mixing within the first fluid conduit;
(e) using the actuator to destroy the blocking member between the first fluid conduit and the second fluid conduit;
(f) using the rotating motor of the driving apparatus to rotate the microfluidic cartridge at a predetermined speed to allow the clinical specimen within the first fluid conduit to flow into the second fluid conduit;
(g) using the rotating motor of the driving apparatus to rotate the microfluidic cartridge at a predetermined speed and angle such that the array of permanent magnets on the driving apparatus interact with a magnetic lysis puck in the second fluid conduit, generating sheer force to break open the cell walls of the cells contained in the clinical specimen and creating a lysate;
(h) using the actuator to destroy the blocking member between the second fluid conduit and the third fluid conduit;
(i) using the rotating motor of the driving apparatus to rotate the microfluidic cartridge at a predetermined speed to allow the clinical specimen lysate within the second fluid conduit to flow into the third fluid conduit;
(j) using the rotating motor of the driving apparatus to oscillate the microfluidic cartridge at a predetermined oscillation frequency and angle to allow a buffer solution containing a series of detector probes, wherein the detector probes are functionalized with a fluorescent signaling molecule, within the third fluid conduit to neutralize the clinical specimen lysate, creating neutralized lysate;
(k) using the actuator to destroy the blocking member between the third fluid conduit and the fourth fluid conduit;
(l) using the rotating motor of the driving apparatus to rotate the microfluidic cartridge at a predetermined speed to allow the neutralized lysate within the third fluid conduit to flow into the fourth fluid conduit;
(m) using the rotating motor of the driving apparatus to rotate the microfluidic cartridge, allowing the neutralized lysate to hybridize and bind with complementary capture probes within the fourth fluid conduit, wherein the capture probes are bound to the surface of a solid surface insert (e.g., plastic);
(n) using the actuator to destroy the blocking member between the wash conduit and the fourth fluid conduit;
(o) using the rotating motor of the driving apparatus to rotate the microfluidic cartridge at a predetermined speed to allow a wash buffer contained in the wash conduit to flow from wash conduit into the fourth fluid conduit;
(p) using the rotating motor of the driving apparatus to position the fourth fluid conduit above the fluorescent detection system in the driving apparatus;
(q) imaging the clinical specimen using a fluorescent microscope; and
(r) using the computer configured with image processing software, to convert the outputting light intensity information from the fluorescent detection system to determine the density of bacteria within the clinical specimen.

In another of its aspects, the present invention provides a method of performing antimicrobial susceptibility testing of a clinical specimen using the system and devices disclosed herein, the method comprising the steps of, the method comprising:
(a) dispensing a predetermined amount of dried-down antibiotics into the first fluid conduit of a microfluidic cartridge;
(b) securing the microfluidic cartridge with respect to the driving apparatus;
(c) dispensing a precise volume of the clinical specimen into the fluid inlet port;
(d) using the rotating motor of the driving apparatus to rotate the microfluidic cartridge at a predetermined speed to allow the clinical specimen within the fluid inlet port to flow into the first fluid conduit;
(e) using the rotating motor of the driving apparatus to oscillate the microfluidic cartridge at a predetermined oscillation frequency and angle to allow turbulent mixing within the first fluid conduit;
(f) using the actuator to destroy the blocking member between the first fluid conduit and the second fluid conduit;
(g) using the rotating motor of the driving apparatus to rotate the microfluidic cartridge at a predetermined speed to allow the clinical specimen within the first fluid conduit to flow into the second fluid conduit;
(h) using the rotating motor of the driving apparatus to rotate the microfluidic cartridge at a predetermined speed and angle such that the array of permanent magnets on the driving apparatus interact with a magnetic lysis puck in the second fluid conduit, generating sheer force to break open the cell walls of the cells contained in the clinical specimen and creating a lysate;
(i) using the actuator to destroy the blocking member between the second fluid conduit and the third fluid conduit;
(j) using the rotating motor of the driving apparatus to rotate the microfluidic cartridge at a predetermined speed to allow the clinical specimen lysate within the second fluid conduit to flow into the third fluid conduit;
(k) using the rotating motor of the driving apparatus to oscillate the microfluidic cartridge at a predetermined oscillation frequency and angle to allow a buffer solution containing a series of detector probes, wherein the detector probes are functionalized with a fluorescent signaling molecule, within the third fluid conduit to neutralize the clinical specimen lysate, creating neutralized lysate;
(l) using the actuator to destroy the blocking member between the third fluid conduit and the fourth fluid conduit;
(m) using the rotating motor of the driving apparatus to rotate the microfluidic cartridge at a predetermined speed to allow the neutralized lysate within the third fluid conduit to flow into the fourth fluid conduit;
(n) using the rotating motor of the driving apparatus to rotate the microfluidic cartridge, allowing the neutralized lysate to hybridize and bind with complementary capture probes within the fourth fluid conduit, wherein the capture probes are bound to the surface of a solid surface insert (e.g., plastic);
(o) using the actuator to destroy the blocking member between the wash conduit and the fourth fluid conduit;
(p) using the rotating motor of the driving apparatus to rotate the microfluidic cartridge at a predetermined speed to allow a wash buffer contained in the wash conduit to flow from wash conduit into the fourth fluid conduit;
(q) using the rotating motor of the driving apparatus to position the fourth fluid conduit above the fluorescent detection system in the driving apparatus;
(r) imaging the clinical specimen using a fluorescent microscope; and
(s) using the computer configured with image processing software, to convert the outputting light intensity information from the fluorescent detection system to determine the density of bacteria within the clinical specimen.

Accordingly, as described herein below, the present inventors have developed a portable microfluidic apparatus, system, and associated methods for biological and analytical testing of biological fluids. More particularly, the invention relates to a rotating cartridge system that is capable of manipulating and analyzing biological fluids.

The devices, systems and methods disclosed herein use microfluidic technology to perform complicated sample-to-answer assays by taking advantage of the unique forces present on a rotating platform to manipulate biological fluids. The disclosed system is capable of performing all of the sample handling and assay steps in both bacteria identification/quantification (IDQ) and Antibiotic Susceptibility Test (AST) assays on a single platform with minimal hardware.

To the knowledge of the inventors, a device for determining bacterial identification and the antimicrobial susceptibility of a microorganism having such a combination of features is heretofore unknown.

Other advantages of the teachings described herein may become apparent to those of skill in the art upon reviewing the present specification.

### BRIEF DESCRIPTION OF THE DRAWINGS

Embodiments of the present invention will be described with reference to the accompanying drawings, wherein like reference numerals denote like parts, and in which:

FIG. 1a illustrates a view of the first surface of one example of a microfluidic cartridge according to various embodiments of the present invention.

FIG. 1b illustrates a view of the second surface of one example of an AST cartridge according to various embodiments of the present invention.

FIG. 2a depicts a schematic diagram of a representative embodiment of the driving apparatus of the present invention according to various embodiments of the present invention.

FIG. 2b depicts a schematic diagram of a representative embodiment of the rotating motor of the present invention according to various embodiments of the present invention.

FIG. 2c depicts a schematic diagram of a representative embodiment of the actuator of the present invention according to various embodiments of the present invention.

FIG. 3 depicts a schematic diagram of a representative embodiment of a rotatable microfluidic cartridge according to various embodiments of the present invention.

FIG. 4a depicts schematic diagram of a representative embodiment of a lysis chamber according to various embodiments of the present invention.

FIG. 4b depicts schematic diagram of a representative embodiment of a neutralization region according to various embodiments of the present invention.

FIG. 4c depicts schematic diagram of a representative embodiment of a detection/hybridization chamber according to various embodiments of the present invention.

FIG. 5 illustrates a detailed view of one example of the valve system on a microfluidic cartridge according to various embodiments of the present invention.

FIG. 6a depicts a schematic diagram of a representative embodiment of a microfluidic cartridge carousel with wedge shaped microfluidic cartridges according to various embodiments of the present invention.

FIG. 6b depicts a schematic diagram of a representative embodiment of a circular disc shaped microfluidic cartridge according to various embodiments of the present invention.

FIG. 6c depicts a schematic diagram of a representative embodiment of fluid inlet port according to various embodiments of the present invention.

FIG. 7a depicts a schematic flow chart of the IDQ process according to various embodiments of the present invention.

FIG. 7b depicts a schematic flow chart of the AST process according to various embodiments of the present invention.

FIG. 8 provides a tabular representation of the steps involved in an example AST process.

FIGS. 9a-m provide a pictorial representation of each step in a particular embodiment of the process of performing IDQ on a clinical specimen on a microfluidic cartridge as disclosed herein.

FIGS. 10a-n provide a pictorial representation of each step in a particular embodiment of the process of performing AST on a clinical specimen on a microfluidic cartridge as disclosed herein.

### DETAILED DESCRIPTION

Various apparatuses or processes will be described below to provide an example of an embodiment of each claimed invention. No embodiment described below limits any claimed invention and any claimed invention may cover processes or apparatuses that differ from those described below. The claimed inventions are not limited to apparatuses or processes having all of the features of any one apparatus or process described below or to features common to multiple or all of the apparatuses described below. It is possible that an apparatus or process described below is not an embodiment of any claimed invention. Any invention disclosed in an apparatus or process described below that is not claimed in this document may be the subject matter of another protective instrument, for example, a continuing patent application, and the applicants, inventors, or owners do not intend to abandon, disclaim, or dedicate to the public any such invention by its disclosure in this document.

The term “specimen” used herein refers to a material which is isolated from its natural environment, including but not limited to biological materials (see definition of “clinical specimen” below), food products, and fermented products.

The term “clinical specimen” used herein refers to clinical specimen or an inoculum derived therefrom, e.g. samples of biological material, including but not limited to urine, blood, serum, plasma, saliva, tears, gastric and/or digestive fluids, stool, mucus, sputum, sweat, earwax, oil, semen, vaginal fluid, glandular secretion, breast milk, synovial fluid, pleural fluid, lymph fluid, amniotic fluid, feces, cerebrospinal fluid, wounds, burns, and tissue homogenates. The clinical specimen may be collected and stored by any means, including in a sterile container. The clinical specimens derived therefrom also include but are not limited to those that are generated in a conventional specimen testing protocol or a traditional laboratory setting.

A clinical specimen may be provided by or taken from any mammal, including but not limited to humans, dogs, cats, murines, simians, farm animals, sport animals, and companion animals.

The term “microbe” used herein refers to any species of microorganism, including but not limited to bacteria, fungi, and parasites.

The term “bacteria” used herein refers to any species of bacteria, including but not limited to Gram-negative and Gram-positive bacteria, anaerobic bacteria, and parasites.

The term “fungi” used herein refers to any species of fungi, including but not limited to the Candida genus, Saccharomyces genus, and Aspergillus genus.

The term “rRNA” used herein refers to the ribosomal ribonucleic acid of bacteria present in the clinical specimen.

The term “rRNA concentration” used herein refers to the number of rRNA molecules per volume tested. rRNA concentration is expressed herein in picomolar (pM) units but can be expressed by any another units.

The term “rRNA signal” used herein refers to the rRNA analyte concentration determined by the quantification of rRNA concentration in a clinical sample. An rRNA signal can be quantified by any known or unknown platform or method. Known platforms include but are not limited to electrochemical sensor platforms, optical platforms (e.g. ELISA, magnetic beads, capture probe arrays), and qRT-PCR.

The term “microbial density” used herein refers to the actual concentration of a given microbe in a specimen. Microbial density is expressed herein in colony forming units per milliliter (CFU/ml) but can be expressed by any another units, including but not limited to genomes per milliliter.

The term “bacterial density” used herein refers to the actual concentration or quantity of bacteria in a specimen. Bacterial density is expressed herein in colony forming units per milliliter (CFU/ml) but can be expressed by any another units, including but not limited to genomes per milliliter.

In some circumstances, time may be of the essence when detecting the presence of bacteria, fungi or other microbes in clinical specimens.

Detection of the presence of certain microbes for example is often the first step in the diagnosis and/or treatment of infectious disease. A given clinical specimen may be been obtained from a subject, whether it be a human or an animal, who may require further medical treatment based on the results of the analysis of the clinical specimen. For example, urine specimens are often obtained from subjects experiencing symptoms consistent with urinary tract infections. Accurately determining the presence of a microbe, or combination of microbes, and preferably a quantum of microbial concentration, in the clinical specimen may help determine an appropriate course of treatment. The goals of such analyses are often to detect possible drug resistance in common pathogens and to assure susceptibility to drugs of choice for particular infections. This information may help clinicians prescribe effective antibiotics or other treatment regimes.

Similarly, in some circumstances, time may also be of the essence when determining the susceptibility of a bacteria, or other microorganism, to an antimicrobial agent.

For example, a given clinical specimen may be obtained from a subject with a suspected infection who may require further medical treatment based on the results of the analysis of the clinical specimen. For example, blood specimens are often obtained from subjects experiencing symptoms consistent with blood sepsis. In these circumstances, it may be desirable to analyze the specimen's response to a variety of different antibiotic agents that could possibly be prescribed to the subject and to determine which of such agents is likely to be relatively more or less effective than the others. For convenience, such analysis would preferably be conducted in a relatively short time period, such as during a routine doctor's visit or in a period of time that the subject might be reasonably expected to wait at the testing location. Preferably, this time period may be less than about 4 hours (or other time limits mentioned herein), and more preferably may be less than about 90 minutes or less than about 60 minutes. This may help a clinician obtain the results while the subject/patient waits, and to then prescribe a desired antibiotic agent for treatment.

Conventional identification/quantification (IDQ) and antimicrobial susceptibility testing (AST) techniques often include at least one growth phase, in which a microbial culture is prepared from the clinical specimen. Such methods may be relatively accurate but may tend to be relatively slow, taking several hours or days to provide useful results to the clinician. In a clinical environment, such time frames may be undesirable and may be considered too long a time period to withhold/delay treatment for a subject. That is, while conventional techniques for determining microbial density and for assessing antimicrobial susceptibility may tend to produce generally accurate results, they may be considered too slow to be of practical assistance. This time delay can sometimes lead to treatments being implemented, such as a particular antibiotic being prescribed, before the bacterial test results are obtained. This may lead to the unnecessary prescription of antibiotics and/or the prescription of a selected antibiotic that is less effective in treating a particular microorganism than other available antibiotics.

In addition to the time required, conventional IDQ and AST techniques often require a skilled technician to set-up and run the microbial cultures, as well as to interpret the results. The analysis may also require specialized and/or costly equipment. As such equipment and skilled technicians can be relatively scarce resources, they are often located in centralized labs and/or hospital environments which are removed from common frontline care facilities, such as a physician's or veterinarian's office, walk-in clinics, and the like. This arrangement can further delay the processing and analysis of clinical specimens by several hours or days, as the specimens must be physically transported from the front-line environment to a centralized testing location and may then wait in a testing queue or backlog of samples awaiting analysis. This time-delay may reduce the accuracy of the ensuing clinical specimen analysis due to such factors as growth or death of any bacteria that may be present in the specimen.

There remains a need for relatively faster specimen analysis methods (in particular for faster IDQ and AST methods), and a need to be able to perform at least some of the analysis in situ in a front-line setting, such as in a physician's or veterinarian's office, instead of having to physically transport the specimens to a centralized location. Similarly, it would be advantageous to provide a method in which a clinically meaningful test result (i.e. information that can help inform treatment decisions) can be provided to a caregiver without requiring the individual skill and judgment of a skilled technician.

To help overcome at least some of these deficiencies in conventional methods of specimen analysis the inventors have developed a new rotatable microfluidic cartridge that can be used, in combination with a suitable test apparatus, for processing and analyzing a clinical specimen.

Preferred embodiments of this apparatus may include any one or a combination of any two or more of any of the following features:
The microfluidic cartridge comprises a body portion configured to be secured with respect to a driving apparatus and configured to be rotated about a rotational axis;
The body portion has a first surface and an opposing second surface;
The microfluidic cartridge has a fluid inlet region configured to receive the clinical specimen;
The microfluidic cartridge has at least a first microfluidic flow path extending downstream from the fluid inlet port,
The microfluidic flow path comprises a series of fluid conduits;
The microfluidic flow path comprises:
   (a) an incubation chamber;
   (b) a lysis chamber;
   (c) a neutralization region;
   (d) a detection/hybridization chamber;
   (e) at least one waste chamber; and
   (f) at least one wash chamber comprising a wash buffer;
wherein (a), (b), (c), (d), (e) and (f) are all connected through a series of microfluidic channels that are configured to allow the clinical specimen to flow from (a) to (b) to (c) to (d) to (e) and to allow the wash buffer to flow from (f) to (d) when the cartridge is rotated.
The incubation chamber is located downstream from the fluid inlet region;
The incubation chamber is configured to receive the clinical specimen from the fluid inlet region when the cartridge is rotated;
The incubation chamber is configured to allow turbulent mixing within the chamber when the cartridge oscillated back and forth along an oscillation path at a predetermined oscillation frequency;
The lysis chamber is connected to and located downstream from the incubation chamber;
The lysis chamber is configured to receive the clinical specimen from the incubation chamber when the cartridge is rotated;
The lysis chamber comprises at least one magnetic lysis puck that can translate within the chamber to cause disruption of a cellular membrane in the clinical specimen when the cartridge is rotated;
The lysis chamber contains at least one chemical lysing agent;
The lysis chamber contains a slurry of ceramic beads, glass beads, zirconium beads, silica-zirconium beads, steel beads or any combination of two or more of these or other chemically inert abrasive microparticles;
The neutralization region is connected to and located downstream from the lysis chamber;
The neutralization region is configured to receive the clinical specimen from the lysis chamber when the cartridge is rotated;
The neutralization region comprises a buffer solution capable of neutralizing the clinical specimen and a series of detector probes;
The detection/hybridization chamber is connected to and located downstream from the neutralization region;
The detection/hybridization chamber is configured to receive the clinical specimen from the neutralization region when the cartridge is rotated; and
The detection/hybridization chamber comprises a series of capture probes, wherein the capture probes are immobilized on a solid surface (e.g., plastic) such that the neutralized clinical specimen is allowed to hybridize and bind with the capture probes when the microfluidic cartridge is oscillated back and forth along an oscillation path at a predetermined oscillation frequency.

In another of its aspects, the present invention relates to a system for processing and analyzing a clinical specimen.

Preferred embodiments of this system may include any one or a combination of any two or more of any of the following features:
The system comprises a driving apparatus with a rotatable motor, an array of permanent magnets, a laser and a fluorescent detection system;
The driving apparatus may be configured to receive a microfluidic cartridge and to rotate the cartridge;
The system comprises a rotatable microfluidic cartridge for processing and analyzing a clinical specimen;
The microfluidic cartridge comprises a body portion configured to be secured with respect to the driving apparatus and configured to be rotated about a rotational axis;
The microfluidic cartridge comprises a fluid inlet region configured to receive the clinical specimen;
The microfluidic cartridge comprises at least a first microfluidic flow path extending downstream from the fluid inlet port;
The microfluidic flow path comprises an incubation chamber located downstream from the fluid inlet region;
The incubation chamber is configured to receive the clinical specimen from the fluid inlet region when the cartridge is rotated;
The incubation chamber is configured to allow turbulent mixing within the chamber when the cartridge oscillated back and forth along an oscillation path at a predetermined oscillation frequency;
The microfluidic flow path comprises a lysis chamber connected to and located downstream from the incubation chamber;
The lysis chamber is configured to receive the clinical specimen from the incubation chamber when the cartridge is rotated;
The lysis chamber comprises at least one magnetic lysis puck that can translate within the chamber to cause disruption of a cellular membrane in the clinical specimen when the cartridge is rotated, and wherein the lysis chamber contains at least one chemical lysing agent;
The microfluidic flow path comprises a neutralization region connected to and located downstream from the lysis chamber;
The neutralization region is configured to receive the clinical specimen from the lysis chamber when the cartridge is rotated;
The neutralization region comprises a buffer solution capable of neutralizing the clinical specimen and a series of detector probes, wherein the detector probes are functionalized with a fluorescent signaling molecule;
The microfluidic flow path comprises a detection/hybridization chamber connected to and located downstream from the neutralization region;
The detection/hybridization chamber is configured to receive the clinical specimen from the neutralization region when the cartridge is rotated; and
The detection/hybridization chamber comprises a series of capture probes, wherein the capture probes are immobilized on a solid surface (e.g., plastic) such that the neutralized clinical specimen is allowed to hybridize and bind with the capture probes when the microfluidic cartridge is oscillated back and forth along an oscillation path at a predetermined oscillation frequency.

In another of its aspects, the present invention relates to a method of performing bacterial identification and quantification (IDQ) on a clinical specimen using the apparatuses and systems disclosed herein.

Preferred embodiments of this IDQ method may include any one or a combination of any two or more of any of the following features:
a microfluidic cartridge is secured with respect to the driving apparatus;
a precise volume of the clinical specimen is dispensed into the fluid inlet port of the microfluidic cartridge;
the rotating motor of the driving apparatus is used to rotate the microfluidic cartridge at a predetermined speed to allow the clinical specimen within the fluid inlet port to flow into the incubation chamber;
the predetermined rotation speed to move the clinical specimen within the fluid inlet port to the incubation chamber is between 3000 RPM and 5000 RPM;
the actuator on the driving apparatus is used to open the valve between the incubation chamber and the lysis chamber;
the rotating motor of the driving apparatus is used to rotate the microfluidic cartridge at a predetermined speed to allow the clinical specimen within the incubation chamber to flow into the lysis chamber;
the predetermined rotation speed to move the clinical specimen from the incubation chamber to the lysis chamber is between 3000 RPM and 5000 RPM;
the rotating motor of the driving apparatus is used to rotate the microfluidic cartridge at a predetermined speed and angle such that the array of permanent magnets on the driving apparatus interact with the magnetic lysis puck in the lysis chamber, generating sheer force to break open the cell walls of the cells contained in the clinical specimen and creating a lysate;
the predetermined rotation speed to lyse the biological is around 200 RPM;
the actuator on the driving apparatus is used to open the valve between the lysis chamber and the neutralization region;
the rotating motor of the driving apparatus is used to rotate the microfluidic cartridge at a predetermined speed to allow the clinical specimen lysate within the lysis chamber to flow into the neutralization region;
the predetermined rotation speed to move the clinical specimen from the lysis chamber to the neutralization region is between 3000 RPM and 5000 RPM;
the rotating motor of the driving apparatus is used to oscillate the microfluidic cartridge at a predetermined oscillation frequency and angle to allow the buffer solution and series of detector probes functionalized with fluorescent signaling molecules within the neutralization region to neutralize the clinical specimen lysate, creating neutralized lysate;
the predetermined oscillation frequency to allow the buffer solution and series of detector probes functionalized with fluorescent signaling molecules within the neutralization region is between 1 Hz and 8 Hz, more preferably about 4 Hz;
the predetermined oscillation angle to allow the buffer solution and series of detector probes functionalized with fluorescent signaling molecules within the neutralization region is about 180 degrees;
the actuator on the driving apparatus is used to open the valve between the neutralization region and the detection/hybridization chamber;
the rotating motor of the driving apparatus is used to rotate the microfluidic cartridge at a predetermined speed to allow the neutralized lysate within the neutralization region to flow into the detection/hybridization chamber;
the predetermined rotation speed to move the clinical specimen from the neutralization region to the detection/hybridization chamber is between 3000 RPM and 5000 RPM;
the rotating motor of the driving apparatus is used to oscillate the microfluidic cartridge at a predetermined oscillation frequency and angle, allowing the neutralized lysate to hybridize and bind with complementary capture probes within the detection/hybridization chamber, wherein the detector probes are bound to the surface of a solid surface insert (e.g., plastic);
the predetermined oscillation frequency to allow the neutralized lysate to hybridize and bind with complementary capture probes within the detection/hybridization chamber is between 1 Hz and 8 Hz, more preferably about 4 Hz;
the predetermined oscillation angle to allow the neutralized lysate to hybridize and bind with complementary capture probes within the detection/hybridization chamber is about 180 degrees;
the actuator on the driving apparatus is used to open the valve between the wash chamber and the detection/hybridization chamber;
the rotating motor of the driving apparatus is used to rotate the microfluidic cartridge at a predetermined speed to allow the wash buffer from the wash chamber to flow from wash chamber;
the predetermined rotation speed to move the wash buffer from the wash chamber to the detection/hybridization chamber is around 1000 RPM;
the rotating motor of the driving apparatus is used to position the detection/hybridization chamber above the fluorescent detection system in the driving apparatus;
the biological fluid sample is imaged using a fluorescent microscope;
the computer configured with image processing software is used to convert the outputting light intensity information from the fluorescent detection system to determine the identity and density of bacteria within the clinical specimen;
the actuator on the driving apparatus is used to open the valve between the detection/hybridization chamber and the waste chamber;
the rotating motor of the driving apparatus is used to rotate the microfluidic cartridge at a predetermined speed to allow the biological fluid in the detection/hybridization chamber to flow from the detection/hybridization chamber into the waste chamber;
the predetermined rotation speed to move the clinical specimen from the detection/hybridization chamber to the waste chamber is between 3000 RPM and 5000 RPM; and
the actuator comprises at least one of a laser and an ultrasound transducer.

In another of its aspects, the present invention relates to a method of performing antimicrobial susceptibility testing (AST) on a clinical specimen using the apparatuses and systems disclosed herein.

Preferred embodiments of this AST method may include any one or a combination of any two or more of any of the following features:
a predetermined amount of an antibiotic is dispensed in the incubation chamber of a microfluidic cartridge;
a microfluidic cartridge is secured with respect to the driving apparatus;
a precise volume of the clinical specimen is dispensed into the fluid inlet port of the microfluidic cartridge;
the rotating motor of the driving apparatus is used to rotate the microfluidic cartridge at a predetermined speed to allow the clinical specimen within the fluid inlet port to flow into the incubation chamber;
the rotating motor system of the driving apparatus is used to oscillate the microfluidic cartridge at a predetermined oscillation frequency and angle to allow turbulent mixing of the clinical specimen with the antibiotic within the incubation chamber;
the predetermined oscillation frequency to allow turbulent mixing of the clinical specimen with the antibiotic within the incubation chamber is between 1 Hz and 8 Hz, more preferably about 4 Hz;
the predetermined oscillation angle to allow turbulent mixing of the clinical specimen with the antibiotic within the incubation chamber is about 180 degrees;
the incubation chamber is incubated;
the time required to incubate the clinical specimen may be from around 1 minute to around 10 hours, more preferably around about 90 minutes;
the incubation step of the AST process may be carried out at a temperature in the range of about range of about 25° C. to 45° C., more preferably around about 37° C.;
the actuator on the driving apparatus is used to open the valve between the incubation chamber and the lysis chamber;
the rotating motor of the driving apparatus is used to rotate the microfluidic cartridge at a predetermined speed to allow the clinical specimen within the incubation chamber to flow into the lysis chamber;
the predetermined rotation speed to move the clinical specimen from the incubation chamber to the lysis chamber is between 3000 RPM and 5000 RPM;
the rotating motor of the driving apparatus is used to rotate the microfluidic cartridge at a predetermined speed and angle such that the array of permanent magnets on the driving apparatus interact with the magnetic lysis puck in the lysis chamber, generating sheer force to break open the cell walls of the cells contained in the clinical specimen and creating a lysate;
the predetermined rotation speed to lyse the biological is around 200 RPM;
the actuator on the driving apparatus is used to open the valve between the lysis chamber and the neutralization region;
the rotating motor of the driving apparatus is used to rotate the microfluidic cartridge at a predetermined speed to allow the clinical specimen lysate within the lysis chamber to flow into the neutralization region;
the predetermined rotation speed to move the clinical specimen from the lysis chamber to the neutralization region is between 3000 RPM and 5000 RPM;
the rotating motor of the driving apparatus is used to oscillate the microfluidic cartridge at a predetermined oscillation frequency and angle to allow the buffer solution and series of detector probes functionalized with fluorescent signaling molecules within the neutralization region to neutralize the clinical specimen lysate, creating neutralized lysate;
the predetermined oscillation frequency to allow the buffer solution and series of detector probes functionalized with fluorescent signaling molecules within the neutralization region is between 1 Hz and 8 Hz, more preferably about 4 Hz;
the predetermined oscillation angle to allow the buffer solution and series of detector probes functionalized with fluorescent signaling molecules within the neutralization region is about 180 degrees;
the actuator on the driving apparatus is used to open the valve between the neutralization region and the detection/hybridization chamber;
the rotating motor of the driving apparatus is used to rotate the microfluidic cartridge at a predetermined speed to allow the neutralized lysate within the neutralization region to flow into the detection/hybridization chamber;
the predetermined rotation speed to move the clinical specimen from the neutralization region to the detection/hybridization chamber is between 3000 RPM and 5000 RPM;
the rotating motor of the driving apparatus is used to oscillate the microfluidic cartridge at a predetermined oscillation frequency and angle, allowing the neutralized lysate to hybridize and bind with complementary capture probes within the detection/hybridization chamber, wherein the detector probes are bound to the surface of a solid surface insert (e.g., plastic);
the predetermined oscillation frequency to allow the neutralized lysate to hybridize and bind with complementary capture probes within the detection/hybridization chamber is between 1 Hz and 8 Hz, more preferably about 4 Hz;
the predetermined oscillation angle to allow the neutralized lysate to hybridize and bind with complementary capture probes within the detection/hybridization chamber is about 180 degrees;
the actuator on the driving apparatus is used to open the valve between the wash chamber and the detection/hybridization chamber;
the rotating motor of the driving apparatus is used to rotate the microfluidic cartridge at a predetermined speed to allow the wash buffer from the wash chamber to flow from wash chamber;
the predetermined rotation speed to move the wash buffer from the wash chamber to the detection/hybridization chamber is around 1000 RPM;
the rotating motor of the driving apparatus is used to position the detection/hybridization chamber above the fluorescent detection system in the driving apparatus;
the biological fluid sample is imaged using a fluorescent microscope;
the computer configured with image processing software is used to convert the outputting light intensity information from the fluorescent detection system to determine the identity and density of bacteria within the clinical specimen;
the computer configured with image processing software is used to test the antibiotic susceptibility of the clinical specimen;
the actuator on the driving apparatus is used to open the valve between the detection/hybridization chamber and the waste chamber;
the rotating motor of the driving apparatus is used to rotate the microfluidic cartridge at a predetermined speed to allow the biological fluid in the detection/hybridization chamber to flow from the detection/hybridization chamber into the waste chamber;
the predetermined rotation speed to move the clinical specimen from the detection/hybridization chamber to the waste chamber is between 3000 RPM and 5000 RPM; and
the actuator comprises at least one of a laser and an ultrasound transducer.

As used herein, certain terms may have the following defined meanings.

As used in the specification and claims, the singular form “a,” “an” and “the” include singular and plural references unless the context clearly dictates otherwise. For example, the term “a cell” includes a single cell as well as a plurality of cells, including mixtures thereof.

In one of its aspects, the invention herein discloses a rotatable microfluidic cartridge for processing and analyzing a clinical specimen. As described herein, the microfluidic cartridge may for example be capable of performing bacteria identification/quantification (IDQ) and/or antimicrobial susceptibility testing (AST) on a clinical specimen.

In certain embodiments, the rotatable microfluidic may comprise a body portion having a first surface and an opposing second surface; a fluid inlet region configured to receive at least one clinical specimen; and at least a first microfluidic flow path extending downstream from the fluid inlet port through the body of the cartridge. In certain preferred embodiments, the microfluidic cartridge may be configured to be secured with respect to a driving apparatus and may be configured to be rotated about a rotational axis when the driving apparatus is in operation.

In certain embodiments, the first microfluidic flow path of the cartridge may comprise two or more fluid conduits connected to one another through a series of microfluidic flow channels that are configured to allow the clinical specimen to flow between the fluid conduits when the cartridge is rotated or oscillated. By way of non-limiting example, the microfluidic cartridge may comprise at least five fluid conduits and one or more wash chambers that are connected to one another through a series of microfluidic flow channels. In certain preferred embodiments, the at least five fluid conduits may include an incubation chamber, a lysing chamber, a neutralization region, a detection/hybridization chamber and a waste chamber.

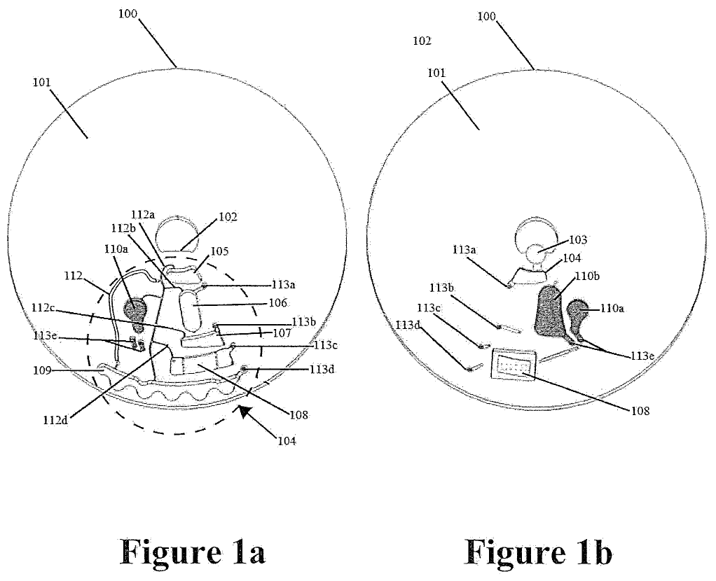

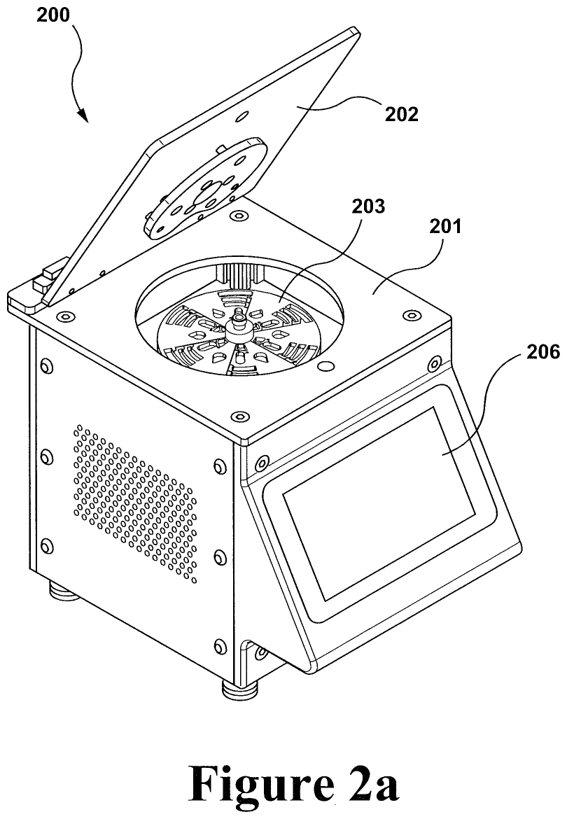

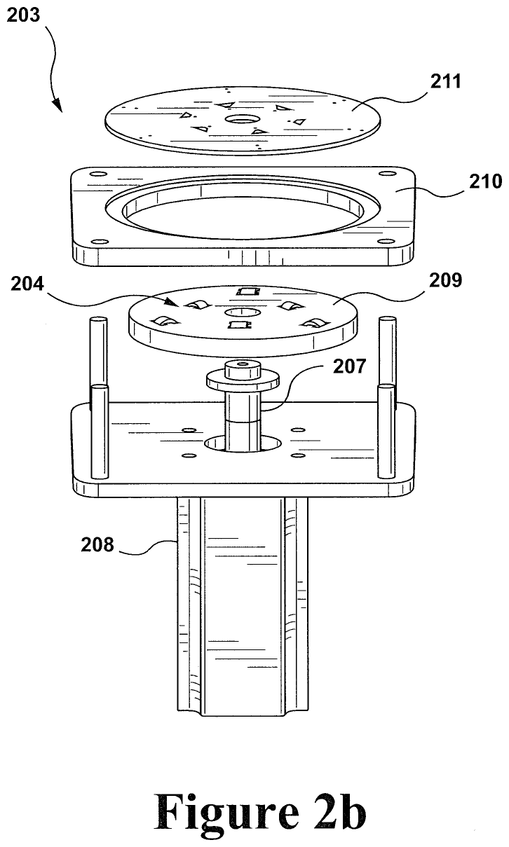

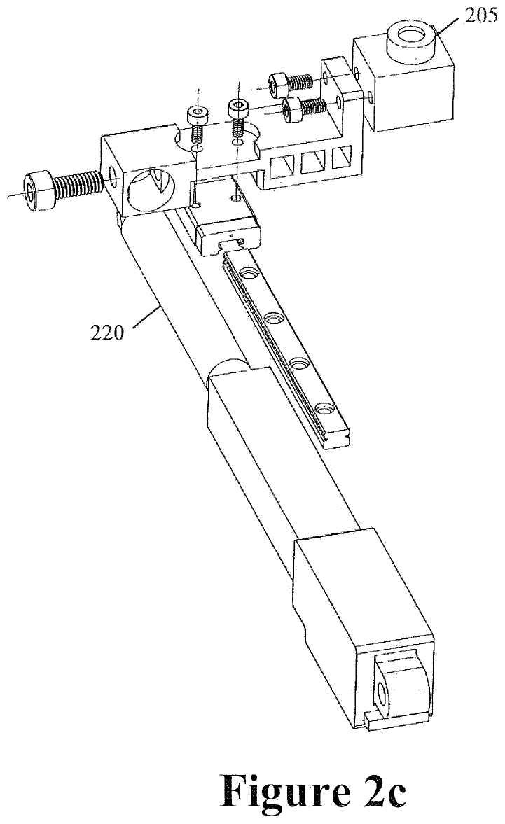

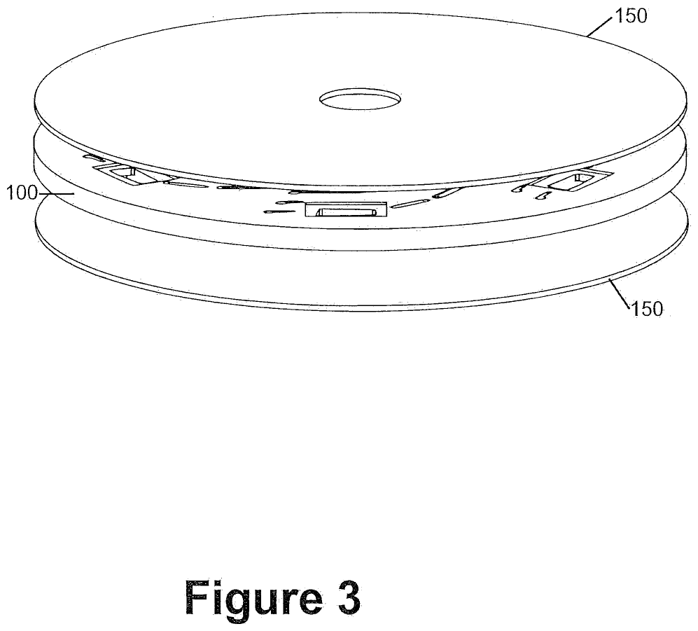

Referring to FIGS. 1a and 1b, one example of a rotatable microfluidic cartridge 100 for processing and analyzing a clinical specimen is provided. FIG. 1a provides a detailed view of the first side of an example microfluidic cartridge 100. FIG. 1b provides a view of an opposing, second side of the microfluidic cartridge 100. As shown in this example embodiment, the microfluidic cartridge 100 may comprise a body portion 101 configured to be secured with respect to a corresponding driving apparatus (see, e.g., 200 FIG. 2a) and configured to be rotated about a rotational axis. By way of non-limiting example, the body portion 101 of the microfluidic cartridge 100 may comprise a securing or alignment portion 102, such as a notch, which may be configured to engage and/or be secured with respect to the driving apparatus. While the embodiment in FIGS. 1a and 1b shows certain components on the first side of the microfluidic cartridge, and other components on the second surface of the microfluidic cartridge, it should be understood that the disclosed invention may include other orientations where any of the disclosed components may be located on either the first or second surface of the microfluidic cartridge. In certain preferred embodiments, the first side of the microfluidic may be sealed with a breathable membrane that can retain liquids within the cartridge while allowing at least some preferred gases to pass through the membranes. Optionally, the second surface of the microfluidic cartridge may be sealed with a breathable membrane. In further embodiments, both the first side and the second surface of the microfluidic cartridge may be sealed with a breathable membrane. A representative view of a microfluidic cartridge which is sealed on both the top/front side and back/bottom side with a breathable membrane is depicted in FIG. 3.

FIG. 3 provides a representative embodiment of the microfluidic cartridge 100 that has been sealed on both the first surface and the second surface with a breathable membrane 150. In certain embodiments, the breathable membrane 150 may be fabricated from a copolymer, such as polyester-polyurethane or polyether-polyurethane. In certain embodiments, the breathable membrane 150 may be comprised of a biocompatible polymer film that is gas permeable and liquid and microbe impermeable. In certain preferred embodiments, the breathable membrane 150 may be configured to permit a flow of gas into and out of one or more fluid conduit on the microfluidic cartridge. This may help facilitate the processes and/or reactions that are to be performed within the microfluidic cartridge, and may allow the venting of waste gases, introduction of oxygen and the like.

As shown in the example embodiment in FIGS. 1a and 1b, the microfluidic cartridge 100 in this example includes a fluid inlet region 103 configured to receive at least one clinical specimen, and at least a first microfluidic flow path 104 extending generally downstream from the fluid inlet region 103. In certain preferred embodiments the microfluidic flow path 104 may include a variety of identifiable chambers or regions that can be configured to facilitate one or more processes or sub-processes. This can help facilitate the performance of a multi-step analysis process using a single microfluidic cartridge and can reduce and or eliminate the need for the clinical specimen to be moved between two or more different apparatuses during the analysis process. This may help facilitate the automated use and/or processing of the clinical specimen and the microfluidic cartridge.

For example, in the illustrated embodiment the microfluidic cartridge 100 includes an incubation chamber 105; a lysis chamber 106; a neutralization region 107; a detection/hybridization chamber 108; and at least one waste chamber 109. The microfluidic flow path 104 may also comprise and at least one wash chamber 110. While a single microfluidic flow path 104 is shown in FIGS. 1a and 1b, the microfluidic cartridge 100 may include two or more microfluidic flow paths 104 that are fluidly isolated from each other, and preferably spaced apart from each other, which may allow several clinical specimens to be processed simultaneously, in parallel with each other on a common microfluidic cartridge 100 (each within a respective one of the microfluidic flow paths 104).

As shown in the preferred embodiment of FIGS. 1a and 1b, the microfluidic cartridge 100 may configured such that the incubation chamber 105, lysis chamber 106, neutralization region 107, detection/hybridization chamber 108 and waste chamber 109 are all connected, in series in this example, through a group of respective microfluidic channels that are configured to allow the clinical specimen to flow from the incubation chamber 105 to the lysis chamber 106 to the neutralization region 107 to the detection/hybridization chamber 108 to the waste chamber 109 when the microfluidic cartridge 100 is rotated. The clinical specimen may be motivated through the channels 104 via the centrifugal forces created by the rotation (in one direction or in a reversing, oscillating style) of the microfluidic cartridge 100. Travel through the microfluidic channels may be controlled by the use of any suitable flow control devices/apparatus and/or valves or other such mechanisms. Preferably, the flow control devices can be actuated in a predetermined manner which may help control the time at which the clinical specimen advances into different portions of the microfluidic cartridge 100, as explained in more detail herein.

The series of microfluidic channels may also be configured to connect the at least one wash chamber 110 to the microfluidic flow path 104, such that a wash buffer in the wash chamber 110 may be allowed to flow from the wash chamber 110 to the any one of the lysis chamber 106, neutralization region 107, or detection/hybridization chamber 108, as desired, when the microfluidic cartridge 100 is rotated and the appropriate flow control mechanisms and/or valves are operated.

In certain embodiments, as depicted in the embodiment of FIGS. 1a and 1b the microfluidic flow path 104 may be configured such that the incubation chamber 105 is located downstream from the fluid inlet region 103. In certain preferred embodiments the incubation chamber 105 may be configured to allow turbulent mixing within the incubation chamber 105 when the cartridge 100 is oscillated back and forth along an oscillation path at a predetermined oscillation frequency.

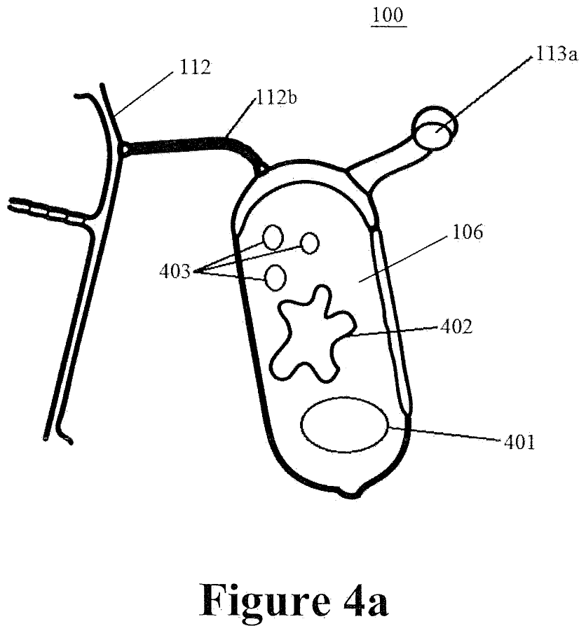

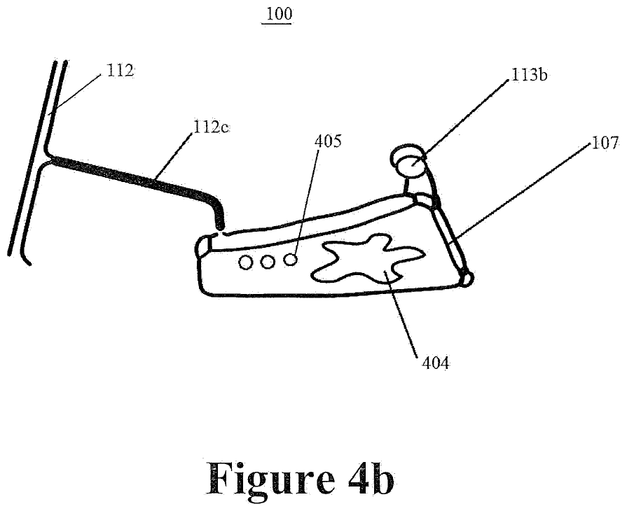

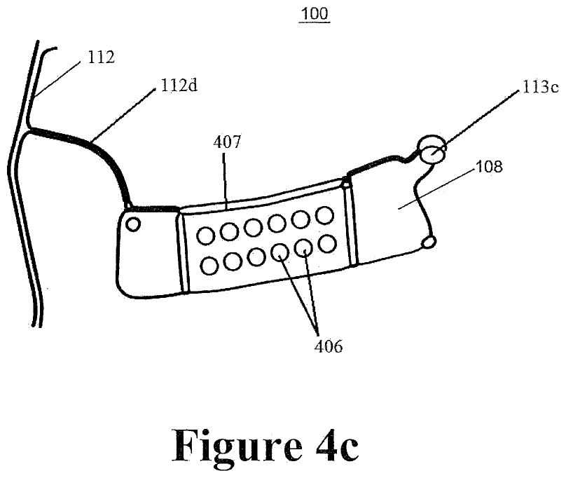

As further depicted in FIGS. 1a and 1b, in certain embodiments, the microfluidic flow path 104 may be configured such that the lysis chamber 106 is connected to and located downstream from the incubation chamber 105. In said embodiments, the lysis chamber may be configured to receive the clinical specimen from the incubation chamber when the cartridge is rotated at a predetermined speed and angle. By way of non-limiting example, the cartridge may be rotated at a speed of around between 3000 RPM to 5000 RPM, or more preferably at a speed of around between 3500 RPM to 4500 RPM, or most preferably at a speed of around 4000 RPM. A schematic representation of one example of a lysis chamber 106 is provided in FIG. 4a.

As further depicted in FIGS. 1a and 1b, in certain embodiments, the microfluidic flow path 104 may be configured such that neutralization region 107 is connected to and located downstream from the lysis chamber 106. In said embodiments, the neutralization region 107 may be configured to receive at least one clinical specimen from the lysis chamber 106 when the cartridge is rotated at a predetermined speed and angle. By way of non-limiting example, the cartridge may be rotated at a speed of around between 3000 RPM to 5000 RPM, or more preferably at a speed of around between 3500 RPM to 4500 RPM, or most preferably at a speed of around 4000 RPM. A detailed schematic view of components of one example of a neutralization region 107 is provided in FIG. 4b.

As further depicted in FIGS. 1a and 1b, in certain embodiments, the microfluidic flow path 104 may be configured such that the detection/hybridization chamber 108 is located downstream from the neutralization region 107. In said embodiments, the detection/hybridization chamber 108 may be configured to receive the clinical specimen from the neutralization region 107 when the cartridge 100 is rotated at a predetermined speed and angle. By way of non-limiting example, the cartridge may be rotated at a speed of around between 3000 RPM to 5000 RPM, or more preferably at a speed of around between 3500 RPM to 4500 RPM, or most preferably at a speed of around 4000 RPM. A detailed schematic view of components of one example of a detection/hybridization chamber 108 is provided in FIG. 4c.

As further depicted in FIG. 1a, in certain embodiments, the microfluidic flow path 104, may optionally include a closed-loop ventilation network 112. The ventilation network 112 may be configured to allow air to move around the microfluidic flow path 104 without the need for vent holes that expose the interior of the microfluidic flow path 104 to the surrounding air. The ventilation network 112 may further be configured so that biological fluid samples cannot enter the ventilation system during operation. By way of non-limiting example, the closed loop ventilation network 112 may comprise a vent line 112a which is connected to and located radially inward from the incubation chamber 105 such that the clinical specimen within the incubation chamber 105 cannot enter the vent line when the cartridge is spinning or oscillating. The closed-loop ventilation network may further comprise a vent line 112b which is connected to and located radially inward from the lysis chamber 106 such that the clinical specimen within the lysis chamber 106 cannot enter the vent line when the cartridge is spinning or oscillating. The closed-loop ventilation network may further comprise a vent line 112c which is connected to and located radially inward from the neutralization region 107 such that the clinical specimen within the neutralization region 107 cannot enter the vent line when the cartridge is spinning or oscillating. The closed-loop ventilation network 112 may further comprise a vent line 112d which is connected to and located radially inward from the detection/hybridization chamber 108 such that the clinical specimen within the detection/hybridization chamber 108 cannot enter the vent line when the cartridge is spinning or oscillating. In certain preferred embodiments, the vent lines of 112a, 112b, 112c and 112d may all be connected as is shown in FIG. 1a.

As further depicted in FIGS. 1a and 1b, in certain embodiments, the microfluidic flow path 104 may further comprise at least one wash chamber 110a connected to and located upstream and radially inward from the neutralization region 107 and detection/hybridization chamber 108. In certain preferred embodiments, the wash chamber 110a may comprise a wash buffer and the detection/hybridization chamber 108 may be configured to receive the wash buffer from the wash chamber 110 when the cartridge is rotated at a predetermined speed, angle and direction. By way of non-limiting example, the cartridge may be rotated at a speed of around between 1000 RPM to 3000 RPM, or more preferably at a speed of around between 1500 RPM to 2500 RPM, or most preferably at a speed of around 2000 RPM.

In certain embodiments, where two wash chambers are used for each microfluidic flow path, the first wash chamber 110a may be milled through the entire thickness of the cartridge 100 while a second wash chamber 110b may be dispensed on the opposite side of the cartridge 100 from the incubation chamber 105, lysis chamber 106, neutralization region 107 and detection/hybridization chamber 108. In certain embodiments, the first and second wash chambers 110a/b may have a specific volume, such that two separate washes may completely fill the detection/hybridization chamber 108 and flow through it, allowing any unbound probes within the detection/hybridization chamber 108 to be washed away. By way of non-limiting example, the first wash chamber 110a may have a volume of about between 200 and 300 μl, or more preferably may have a volume of about between 250 and 275 μl, or most preferably may have a volume of about 255 μl. In certain embodiments, the second wash chamber 110a may have a slightly higher volume than the first wash chamber 110a. By way of non-limiting example, the second wash chamber 110b may have a volume of about between 200 and 300 μl, or more preferably may have a volume of about between 250 and 275 μl, or most preferably may have a volume of about 259 μl.

In certain embodiments, the one or more wash chambers 110 may be shaped to include angled corners to allow for easy emptying of the wash chamber. Further, in certain embodiments the one or more wash chambers 110 may be separated such that there may be a spin dry step between washes.

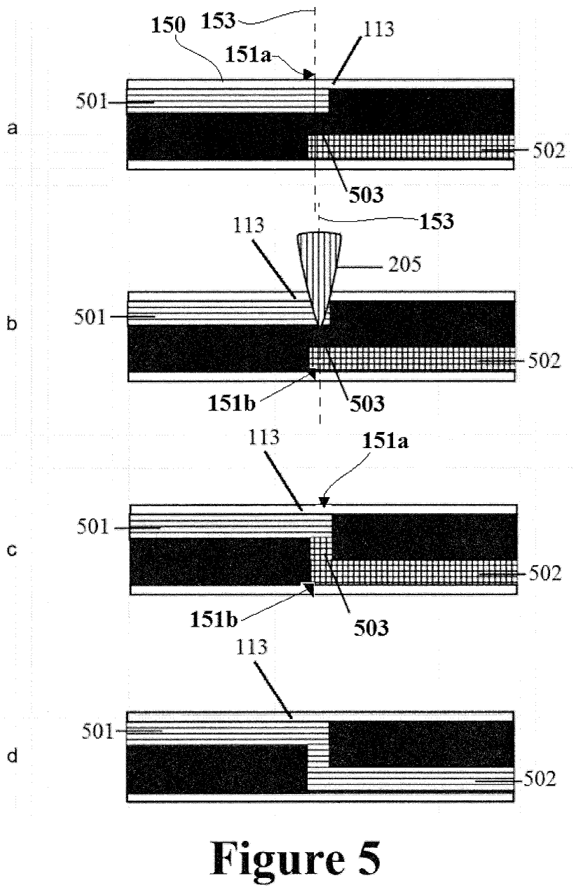

As further depicted in FIGS. 1a and 1b, in certain preferred embodiments, the microfluidic flow path 104 may further comprise one or more valves 113 which make up a valving system. In certain preferred embodiments, these one or more valves 113 may be used as a gate between different chambers on the microfluidic cartridge 100. By way of non-limiting example, the one or more valves 113 may be configured to restrict or allow the movement of the clinical specimen within the microfluidic flow path 104 until opened or actuated a pre-determined time or step in the overall analysis process. In certain preferred embodiments, each of the one or more valves 113 within the valving system may comprise a blocking member positioned in the microfluidic flow channel between two fluid conduits and fluidly isolating the two fluid conduits from one another. In certain preferred examples, the blocking member being spaced inwardly from the first and second surface of the body of the microfluidic cartridge 100. The blocking member may be configured to be at least partially destroyable via an actuator 205 (see FIG. 2c) that is external to the microfluidic cartridge 100 when the cartridge is in use, thereby establishing fluid communication between two fluid conduits at a predetermined time and allowing the clinical specimen to flow between fluid conduits. By way of non-limiting example, the blocking member may be provided in the form of a frangible and/or destroyable plastic member covering between two microfluidic flow channels on opposite sides of the microfluidic cartridge 100 (see FIG. 5).

In certain preferred embodiments, each of the one or more valves 113 within the valving system may be manipulated by a suitable actuator 205. Optionally, the actuator 205 may be external the microfluidic cartridge 100 but can still be configured to engage and trigger the valves 113 within the microfluidic cartridge 100. In some arrangements the valves 113 themselves may be relatively static/passive members and the movable and/or active actuator can be provided on the driving apparatus 200 or other suitable apparatus. This may help simplify the construction of the microfluidic cartridge 100. This may also help facilitate the use of a common actuator 205 to trigger the valves 113 on multiple, different microfluidic cartridges 100. The actuator 205 may be of any suitable construction and may include any suitable mechanism to engage the valves 113, including a mechanical piercing or lancing member, a laser or other light-based mechanism, ultrasonic or other sound-based mechanisms and the like.

In the illustrated embodiment the actuator 205 is located on the driving apparatus 200. To open any one of the valves 113, the actuator may be configured to destroy the blocking member between two fluid conduits, thereby establishing fluid communication between the two or more fluid conduits at a predetermined time and allowing the clinical specimen to flow from one fluid conduit into another fluid conduit when the microfluidic cartridge is rotated or oscillated. In certain preferred embodiments, the actuator 205 may be a laser that is configured to melt the blocking member between two fluid conduits, while not melting the outer layers of the microfluidic cartridge 100. This can allow the valve 113 to be triggered without rupturing the flow path 104 (i.e. to prevent leakage of the clinical specimen out of the flow path 104) and/or damaging other portions of the microfluidic cartridge 100 that do not need to be altered to trigger the valve 113.

In certain preferred embodiments, there may be a first valve 113a between the incubation chamber 105 and the lysis chamber 106. There may also be a second valve 113b between the lysis chamber 106 and the neutralization region 107. There may also be a third valve 113c between the neutralization region 107 and the detection/hybridization chamber 108. There may also be a fourth valve 113d between the wash chamber and the detection/hybridization chamber and a valve between the detection/hybridization chamber and the waste chamber.

The steps of the procedure to open any of the valves 113 is depicted graphically in FIGS. 5a-d. FIG. 5a provides a representative diagram of the cross section of a valve 113 between two example fluid conduits, 501 and 502, of a microfluidic cartridge 100. As shown in FIG. 5a, the valve 113 may comprise a blocking member 503 positioned in the microfluidic flow channel between two fluid conduits and fluidly isolating the two fluid conduits 501 and 502 from one another. As shown in FIG. 5b, in order to manipulate the valve 113, an actuator 205 (see FIG. 2c) may be positioned over the blocking member 503 and may at least partially destroy the blocking member 503 between two fluid conduits 501 and 502. As shown in FIG. 5c, once the blocking member 503 has been destroyed by the actuator 503, fluid communication may be established between the two fluid conduits 501 and 502. As shown in FIG. 5d, once this fluid communication is established, fluid from one conduit (501) may flow into the other conduit (502) when the microfluidic cartridge is rotated or oscillated.

In embodiments where the actuator 205 includes a laser or other light-based mechanism, the light beam may preferably pass through the breathable membrane 150 that helps bound the conduit 501. The region through which the light beam passes can be referred to as a transmission portion 151a of the membrane 150 that is configured to help facilitate the passage/transmission of the light beam without itself becoming melted or otherwise damaged. In contrast, the blocking member 503 is preferably configured to absorb and/or react with the light beam in such a manner that the blocking member 503 can be melted when needed. In the present embodiment, this is facilitated by configuring the transmission portion 151a to have a relatively higher optical transmissivity than the blocking member 503 (i.e. to absorb less energy from the light beam). For example, the transmission portion 151a may be substantially transparent.

Preferably, the incoming light beam, or other such actuator member can be arranged to travel along an actuator axis, a laser axis 153 in FIG. 5 to help target/focus the actuator. Preferably, the transmission portion 151a is positioned so that it is axially between the actuator 205 (i.e. source of the light beam) and the blocking member 503. That is, the transmission portion 151a is preferably registered above the blocking member 503 (i.e. overlies the blocking member 503 when in use). Optionally, another transmission portion 151b may be positioned axially on the other side the blocking member 503. In this arrangement, the laser 205 can be used to melt the blocking member 503 and any portions of the light beam that pass through the blocking member 503 (or the space where the blocking member 503 used to be) can extend downwardly through the microfluidic cartridge 100 and exit via the lower or second transmission portion 151b, instead of hitting other portions of the cartridge body 101 that may be prone to damage by the laser 205. This can reduce the chances of the cartridge body 101 being damaged by the laser 205 when the blocking member 503 is being melted and may help reduce the degree of precision required when timing the firing time of the laser 205, as any “over spray” of the laser is unlikely to damage the microfluidic cartridge 100. This may also help facilitate the use of a sensor or other such apparatus on the opposite side of the cartridge 100 from the laser 205 as a mechanism to help sense/detect when the blocking member 503 has been melted (i.e. by detecting laser light that can now pass thorough the cartridge 100 and reach the underlying sensor). To help reduce the overall size and complexity of the microfluidic cartridge 100, one or both of the transmission portions 151a and 151b can also be functional portions of the membranes 150 and can help bound the channels 501 and 502. Optionally, the entire membranes 150 can be configured to have the desired properties of the transmission portions 151a and 151b (and may therefore be of integral, one-piece construction), or alternatively the transmission portions 151a and 151b may be made of a different material and/or have different properties than other portions of the membranes 150.

Preferably, the blocking member 503 is the only, or substantially the only, portion of the body 101 of the cartridge 100 that is positioned axially between the upper transmission portion 151 and the lower transmission portion 151b along the actuator axis 153. That is, the blocking member 503 may be the only non-transparent (or sufficiently low optical transmissive) portion of the microfluidic cartridge 100 that is intersected by the actuator axis 153. Optionally, the blocking member 503 may be integrally formed with other portions of the body 101, and/or the body 101 may be entirely formed of integral, one-piece construction (such as being molded as a single plastic piece). In such an arrangement, substantially all of the body 101 and its features (flow channels, chambers, etc.) would be meltable by the actuator 205. Arranging the blocking member 503 and transmission portions 151a and 151b in the manner shown in FIG. 5, in which the blocking member 503 is the only part of the body 101 that is hit by the laser light beam, may help facilitate the one-piece construction of the body 101 while still allowing the local melting of the blocking member(s) 503 on demand.

Similarly, to help ensure only desired portions of the body 101 are melted, the valves 113 and particularly the blocking members 503 can be physically, laterally spaced apart from other sensitive and/or important portions of the cartridge 100, such as the chambers, inlets and other structures. A similar configuration can be used proximate each of the blocking members 503 used in each of the valves 113 in a given cartridge 100.

Using a laser 205 can help facilitate melting/destruction of the blocking members 503 without requiring a mechanical or direct physical engagement between the external actuator and the valves 113 in the cartridge 100. This may help preserve the desired liquid tight configuration of the cartridge 100 while valves 113 are sequentially actuated as the cartridge 100 is in use.

### Lysis Chamber

FIG. 4a provides a detailed depiction of the components of one embodiment of a lysis chamber 106. As shown in FIG. 4a, in certain embodiments, the lysis chamber 106 may comprise at least one magnetic lysis puck 401 that may translate within the lysis chamber 106 to cause disruption of a cellular membrane in the clinical specimen when the cartridge is rotated. In certain preferred embodiments, the lysis chamber 106 may also contain at least one chemical lysing agent 402.

In certain embodiments, each lysis chamber may be filled with a slurry of silica-zirconia beads 403 and at least one magnetic lysis puck 401. In said embodiment, when the microfluidic cartridge is rotated via the driving apparatus 200, an array of permanent magnets (see 204 FIG. 2b) on the driving apparatus 200 may interact with the magnetic lysis puck 401 in the lysis chamber 106 such that the lysis puck 401 may grind the silica-zirconia beads within the lysis chamber 106, generating shear force to break open the cell walls of any clinical specimen contained within the lysis chamber 106. In further embodiments, each lysis chamber may contain a chemical lysing agent 402, for example, a dried down alkaline buffer such as NaOH which may allow for chemical lysis of any clinical specimen contained within the lysis chamber 106. In certain embodiments, the alkaline buffer within the lysis chamber 106 may be dried down inside a divot in the COC and may be covered with paraffin wax so that the alkaline buffer remains stable until it is combined with any clinical specimen entering the lysis chamber 106.

In certain embodiments, the lysis chamber 106 may be oriented parallel with the radius of the microfluidic cartridge 100 and may be long enough to allow the lysis puck 401 to travel a sufficient distance within the lysis chamber 106 to increase interactions between the silica-zirconia beads 403 and the lysis puck 401. Alternatively, in certain embodiments, the lysis chamber 106 may be oriented perpendicular to the radius of the microfluidic cartridge 100 in order to save space on the cartridge.

In certain embodiments, each lysis puck 401 in the lysis chamber 106 may be made of any ferrous metal. Further, in certain embodiments, each lysis puck 401 in the lysis chamber 106 may be about the same width as the lysis chamber 106.

In certain embodiments, the microfluidic flow path leading into the lysis chamber may have a diameter less than that of the diameter of the silica-zirconia beads 403, such that the silica-zirconia beads 403 cannot leave the lysis chamber 106. By way of non-limiting example, in certain embodiments, the silica-zirconia beads may have a diameter of about 100 microns and the microfluidic path channel leading into the lysis chamber may have a diameter less than 100 microns. In other embodiments, a filter may be used to prevent the silica-zirconia beads 403 from leaving the lysis chamber 106.

In certain embodiments, the valve 113a between the incubation chamber 105 and the lysis chamber 106 may be located far enough away from the lysis chamber 106 such that the actuator 205 (see FIG. 2c) of the driving apparatus 200 will not interact with the lysis puck 401 within the lysis chamber 106.

In certain embodiments, the lysis chamber 106 will have a volume large enough to accommodate at least one lysis puck 401, at least about 70 μl of slurry silica-zirconia beads 403, at least about 1M of dried down chemical lysing agent (e.g., NaOH) 402, and at least about 150 μl of biological fluid sample. By way of non-limiting example, in certain embodiments, the lysis chamber may have a volume of at least about between 200 and 250 μl, more preferably of about between 205 and 210 μl, or most preferably of about 208 μl.

### Neutralization Region

FIG. 4b provides a detailed depiction of the components of one embodiment of a neutralization region 107. As shown in FIG. 107, in certain embodiments, the neutralization region 107 may comprise a buffer solution 404 and a series of detector probes functionalized with fluorescent signaling molecules 405, wherein the buffer solution and/or the detector probes may be dried in several places inside the neutralization region 107 to allow for resuspension of a dried down reagent. By way of non-limiting example, the buffer solution 404 may be a phosphate buffer solution.

In certain embodiments, the neutralization region 107 may comprise a wide angular span to allow for enhanced mixing during oscillation. In further embodiments, the neutralization region 107 (or other chambers including the incubation chamber, etc.) may comprise angled corners to allow the clinical specimen to be easily emptied from the neutralization region 107 following neutralization. Alternatively, in certain embodiment the neutralization region 107 may be a serpentine-shaped channel with multiple bends, wherein the buffer solution 404 and detector probes 405 may be dried down in various positions on the first two bends of the serpentine-shaped channel.

In certain embodiments, the neutralization region 107 may have a volume slightly larger than the biological fluid sample being analyzed by the system to allow for the sample to fill the neutralization region 107 and resuspend the buffer solution 404 while allowing for oscillatory mixing. By way of non-limiting example, in certain embodiments, where a biological fluid sample of about 150 μl is used, the volume of the neutralization region 107 may be at least about 1554

### Detection/Hybridization Chamber

FIG. 4c provides a detailed depiction of the components of one embodiment of a detection/hybridization chamber 108. As shown in FIG. 4c, in certain embodiments, the detection/hybridization chamber 108 may comprise a series of capture probes 406, where said capture probes 406 are immobilized on a solid surface (e.g., plastic) such that a neutralized clinical specimen from the neutralization region 107 may be allowed to hybridize and bind with the capture probes 406 when the microfluidic cartridge 100 is oscillated back and forth along an oscillation path at a predetermined oscillation frequency.

In certain embodiments, the detection/hybridization chamber 108 may contain a solid surface insert (e.g., a plastic insert). Further, in certain embodiments, that detection/hybridization chamber 108 may comprise a series of spotted down capture probes, wherein the capture probes are bound to the surface of the solid surface insert such that when the cartridge is oscillated/rotated by the motor of the driving apparatus, the neutralized biological fluid sample is allowed to hybridize and bind with the capture probes. In certain embodiments, the detection/hybridization chamber comprises a viewing window 407 that may be the same size as the viewing area of a monochrome camera in a fluorescent detection system of the driving apparatus 200 such that the entire window 407 may be imaged by the camera at once. Alternatively, in certain embodiments, there may be multiple cameras within the fluorescent detection system of the driving apparatus 200.

In certain embodiments, the detection/hybridization chamber 108 may comprise a wash inlet and a waste outlet, wherein the wash inlet and waste outlet are on opposite ends of the chamber, allowing for a flow through wash within the detection/hybridization chamber 107.

In certain embodiments, the detection/hybridization chamber 108 may have a long span allowing for mixing during hybridization.

In certain embodiments, the detection/hybridization chamber 108 may have a volume large enough to completely submerge the capture probes 406 at all times while still having a small amount of air to allow for light oscillation during hybridization. By way of non-limiting example, where a biological fluid sample of about 150 μl is used, the volume of the detection/hybridization chamber may be at least about 152 μl.

In certain embodiments, the body 101 of the microfluidic cartridge 100 may be made of dark coloured non-toxic material that can withstand lasers. By way of non-limiting example, in certain embodiments, the body of the cartridge may be made of, for example, black cyclic olefin copolymer (COC). Alternatively, in other embodiments, the body 101 of the microfluidic cartridge 100 may be made of, for example, black PMMA (acrylic). In certain embodiments, the body of each cartridge may be covered on the front and back side with transparent adhesive layer, allowing lasers to pass through the cartridge.

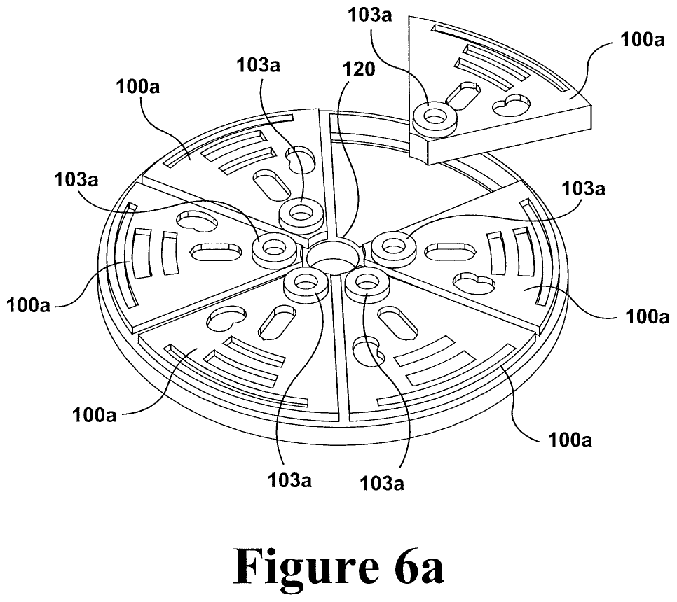

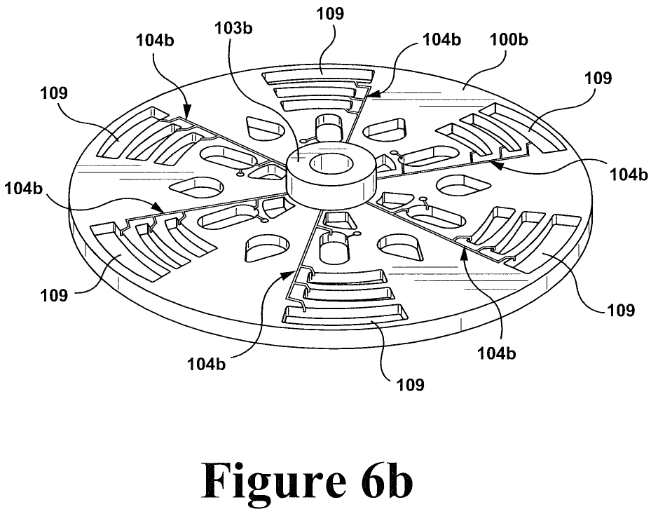

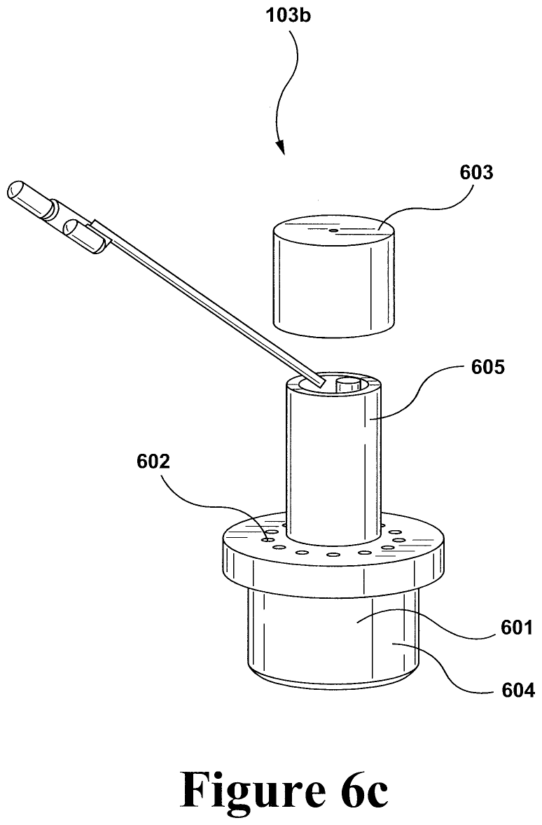

In certain embodiments, the body of each microfluidic cartridge 100a may by shaped like a wedge or pie piece and may be configured to fit along with a series of other wedge-shaped cartridges in a round carousel. (see, e.g., FIG. 6a). In said embodiment, as shown in FIG. 6a, the carousel 120 may be configured to be loaded onto the rotating motor 207 of the driving apparatus 200. By way of non-limiting example, in certain embodiments, each carousel 120 may be configured to hold up to six (6) microfluidic cartridges 100a, wherein each cartridge may comprise at least one microfluidic flow path 104a configured to receive and process biological fluid samples, such that each carousel may be used to analyze up to six (6) clinical specimens at one time. In certain embodiments, when wedge-shaped cartridges are used, each wedge-shaped cartridge may comprise one fluid input port 103a, such that, for instance, when a carousel 120 contains six cartridges 100a, each cartridge has a unique fluid entry port.

Alternatively, in certain embodiments, the body of each cartridge may be shaped like a round disc (see, e.g., FIG. 6b) and may be configured to be loaded directly onto the rotating motor 207 of the driving apparatus 200 without the need for a carousel 120. In said embodiment, the cartridge 100b may comprise multiple microfluidic flow paths 104b on one disc-shaped cartridge 100b, wherein each microfluidic flow path 104b shares a common fluid input port 103b. By way of non-limiting example, in certain embodiments, each cartridge 100b may be configured to comprise up to six (6) microfluidic flow paths 104 configured to receive and process biological fluid samples from a common fluid inlet port 103b, such that each cartridge may be used to run up to six (6) tests at one time. In certain embodiments, each microfluidic path on a disc-shaped cartridge 100b may comprise its own waste chamber 109. Alternatively, in certain embodiments, the waste chambers of each microfluidic flow path 104 on a disc-shaped cartridge 100b may be connected to create only one waste chamber 109 per cartridge 100b.

A representative example of an embodiments of this common fluid inlet port 103b is depicted in FIG. 6c. As depicted in FIG. 6c, in certain preferred embodiments, the common fluid inlet port 103b of a disc shaped microfluidic cartridge 100b may contain a sample entry cone 601, disc interface channels 602 and a piercing cap 603. In certain preferred embodiments, the common fluid inlet port 103b may comprise two buffer pouches: a 1:10 dilution buffer pouch 604 that is always opened as soon as the fluid inlet port 103b is placed onto microfluidic cartridge 100 and is located in the sample entry cone 601; and a 1:100 dilution buffer pouch 605 that is opened only if necessary via a programmable plunger in the piercing cap 603. After the correct volume of sample is pipetted into the common fluid inlet port 103b and the proper dilution buffer packages are broken, the motor system 203 of the driving apparatus 200 (see FIGS. 2a, 2b) may be oscillated back and forth in order to mix the clinical specimen with the dilution buffer.

In certain embodiments, each microfluidic path on a cartridge may include a series of overflow channels, such that any excess fluid within any chamber of the microfluidic flow path will run off to the waste chamber via said overflow channel.

### Driving Apparatus

Turning now to FIG. 2a an example embodiment of a driving apparatus 200 configured for use with the system disclosed herein for processing and analyzing a clinical specimen is provided.

As shown in FIG. 2a, in certain embodiments, the driving apparatus 200 may comprise the following components: an outer shell 201; a lid 202; a rotating motor 203; an array of permanent magnets 204; at least one actuator (see 205, FIG. 2c); and a touchscreen interface 206. In certain embodiments, the driving apparatus 200 may further comprise any one of or combination of: a temperature control system; a fluorescent detection system; a safety switch; and an active cooling system. The array of permanent magnets 204 may be configured to generate a stationary magnetic field that may be used in conjunction with a magnetic steel disc or lysis puck 401 in the lysing chamber 106 for mechanical lysis (see, e.g., FIG. 4a). This puck 401 can be moved back and forth by outside stationary magnets located in the drive apparatus to grind the silica zirconia beads generating shear forces to lyse bacterial or fungal cells.

In certain embodiments, the touchscreen interface 206 may be used to control the driving apparatus 200, to process fluorescent images, and to troubleshoot the driving apparatus 200. In certain embodiments, the safety switch may be built into the lid 202 of the driving apparatus 200 and said safety switch may be configured to prevent an operator of the driving apparatus 200 from being exposed to harmful lasers. In certain embodiments, the fluorescent detection system of the driving apparatus 200 may comprise a monochrome camera, at least one excitation filter, at least one emission filter, a dichroic mirror, and a series of excitation LEDs. In certain embodiments, the temperature control system of the driving apparatus 200 may be used to maintain a desired temperature within the driving apparatus. It may desirable for example to keep the temperature within the driving apparatus 200 at or around about 37° C., as this is an optimal temperature for bacterial growth. By way of non-limiting example, the temperature control system may comprise a heater, a series of blowers, and a thermocouple. In certain embodiments, the cooling system of the driving apparatus 200 may comprise an integrated set of fans and heat sinks that may be configured to prevent the driving apparatus from overheating.

A detailed view of one example embodiment of the rotating motor system 203 of the driving apparatus 200 is provided in FIG. 2b. In certain embodiments, the rotating motor system 203 may comprise a motor 207, a motor stand 208, a magnet holder 209 on which the array of permanent magnets 204 is arranged, a disc holder 210, and a rotating disc 211, wherein the rotating disc 211 may be configured to secure a microfluidic cartridge 100. By way of non-limiting example the securing portion 102 of the microfluidic disc may be configured to interface with the another securing portion on the rotating disc 211, such that when the microfluidic cartridge 100 is housed on the rotating disc 211 and the rotating motor system 207 is turned, oscillated, or rotated, the microfluidic cartridge 100 will remain on the rotating disc 211 and will oscillate or rotate in unison with the rotating disc 211 at a predetermined speed, interval and angle.

In certain embodiments, the magnet holder 209 may be round and may house an array of permanent magnets 204 secured to the outer and inner radii of the magnet holder 209 such that, as the motor 203 rotates the rotating disc 211, the magnetic lysis puck 401 within the lysis chamber 106 in the at least one microfluidic flow path 104 on the microfluidic cartridge 100 moves back and forth within the lysis chamber 106 as it is alternately attracted to the permanent magnets 204 on the magnet holder 209.

FIG. 2c provides a detailed view of one embodiment of the actuator 205 which may be part of the driving apparatus 200 and may be configured to manipulate the valves 113 of a microfluidic cartridge 100. In certain preferred embodiments, the at least one actuator 205 of the driving apparatus 200 may be used to control the flow of a biological fluid sample on a microfluidic cartridge 100. By way of non-limiting example, the at least one actuator 205 may be connected to the driving apparatus by a control arm 220. By way of non-limiting example, the control arm 220 may include a linear actuator or other suitable apparatus that can be configured for radial position control (i.e. to change the radial position of the actuator 205 relative to the microfluidic cartridge 100). Radial movement of this type, combined with a controller rotation of the microfluidic cartridge 100 about its rotation axis may facilitate the positioning of the actuator 205 in a variety of desired and/or pre-determined positions relative to the microfluidic cartridge 100, which may help align a given actuator 205 with different ones of the valves 113 at different times.

As discussed herein, the actuator 205 may be configured to open any one of the valves 113 of a microfluidic flow path 104 on a microfluidic cartridge 100. To open a valve 113, the actuator 205 may be focused on the valve position and may destroy the blocking member between to fluid conduits in the microfluidic flow path 104 of a microfluidic cartridge 100, allowing free flow of liquid between the conduits. By way of non-limiting example, positioning of the actuator 205 may be accomplished by: setting the radial position with a servo motor and linear rail onto which the actuator 205 is mounted; and setting the angular position of the actuator 205 by moving the servo motor to a specified angular position.

In certain preferred embodiments, the actuator 205 may be a laser device. In said embodiments, when the laser is positioned above any valves 113, it may be turned on and may melt the blocking member in between two fluid conduits in a microfluidic flow path 104 of a microfluidic cartridge, allowing free flow of liquid between the conduits.

### Process/Methods

### IDQ

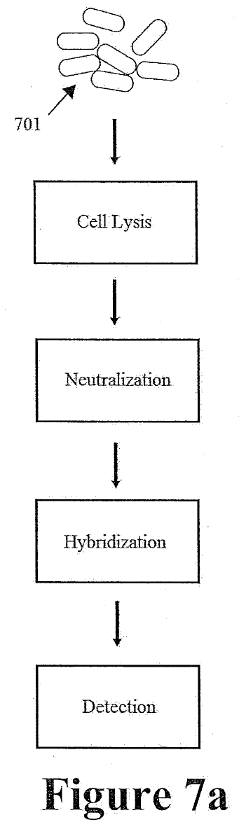

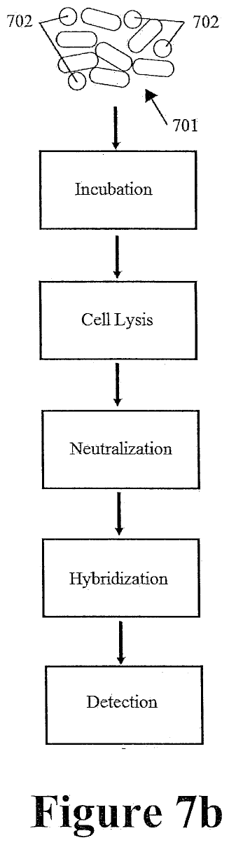

In yet another of its aspects, the invention herein discloses a method of performing bacterial identification and quantification (IDQ) on a clinical specimen 701, using the system described herein (see, e.g., FIG. 7a). As shown in FIG. 7a, the method for performing IDQ may comprise the steps of sample entry, cell lysis, neutralization, hybridization, and detection.

In certain embodiments, the method of performing IDQ involves the use of a microfluidic cartridge 100, as shown for example in FIGS. 1a and 1b and the use of a driving apparatus, as shown for example in FIGS. 2a-c. In certain preferred embodiments, the method of performing IDQ may comprise the steps of:
(a) securing the microfluidic cartridge 100 with respect to the driving apparatus 200;
(b) dispensing a precise volume of the clinical specimen 701 into the fluid inlet port 103;
(c) using the rotating motor system 203 of the driving apparatus 200 to rotate the microfluidic cartridge 100 at a predetermined speed to allow the clinical specimen within the fluid inlet port 103 to flow into the incubation chamber 105;
(d) using the actuator 205 to open the valve 113a between the incubation chamber 105 and the lysis chamber 106;
(e) using the rotating motor system 203 of the driving apparatus 200 to rotate the microfluidic cartridge 100 at a predetermined speed to allow the clinical specimen within the incubation chamber 105 to flow into the lysis chamber 106;
(f) using the rotating motor system 203 of the driving apparatus 200 to rotate the microfluidic cartridge 100 at a predetermined speed and angle such that the array of permanent magnets 204 on the driving apparatus 200 interact with the magnetic lysis puck 401 and the chemical lysing agent 402 in the lysis chamber 106, generating sheer force to break open the cell walls of the microorganisms and chemically degrade the cells and their biological material contained in the clinical specimen and creating a lysate;
(g) using the actuator 205 to open the valve 113b between the lysis chamber 106 and the neutralization region 107;
(h) using the rotating motor system 203 of the driving apparatus 200 to rotate the microfluidic cartridge 100 at a predetermined speed to allow the clinical specimen lysate within the lysis chamber 106 to flow into the neutralization region 107;
(i) using the rotating motor system 203 of the driving apparatus 200 to oscillate the microfluidic cartridge 100 at a predetermined oscillation frequency and angle to allow the buffer solution 404 and a series of detector probes functionalized with fluorescent signaling molecules 405 within the neutralization region 107 to neutralize the clinical specimen lysate, creating neutralized lysate;
(j) using the actuator 205 to open the valve 113c between the neutralization region 107 and the detection/hybridization chamber 108;
(k) using the rotating motor system 203 of the driving apparatus 200 to rotate the microfluidic cartridge 100 at a predetermined speed to allow the neutralized lysate within the neutralization region 107 flow into the detection/hybridization chamber 108;
(l) using the rotating motor system 203 of the driving apparatus 200 to oscillate the microfluidic cartridge 100, allowing the neutralized lysate to hybridize and bind with complementary capture probes 406 within the detection/hybridization chamber 108, wherein the capture probes 406 are bound to the surface of a solid surface insert (e.g., plastic);
(m) using the actuator 205 to open the valve 113d between the detection/hybridization chamber 108 and the waste chamber 109;
(n) using the rotating motor system 203 of the driving apparatus 200 to rotate the microfluidic cartridge 100 at a predetermined speed to allow the biological fluid in the detection/hybridization chamber 108 to flow from the detection/hybridization chamber 108 into the waste chamber 109;
(o) using the actuator 205 to open the valve 113e between the wash chamber 110a and the detection/hybridization chamber 108;
(p) using the rotating motor system 203 of the driving apparatus 200 to rotate the microfluidic cartridge 100 at a predetermined speed to allow the wash buffer from the wash chamber 110a to flow from wash chamber 110a and into the detection/hybridization chamber 108;
(q) using the rotating motor system 203 of the driving apparatus 200 to position the detection/hybridization chamber 108 above the fluorescent detection system in the driving apparatus 200;
(r) imaging the biological fluid sample using a fluorescent microscope; and
(s) using the computer configured with image processing software, to convert the outputting light intensity information from the fluorescent detection system to determine the density of bacteria within the clinical specimen.

In certain embodiments, the method may further comprise the steps of:

In certain embodiments, the process for bacterial identification and quantification (IDQ) on a microfluidic cartridge such as those described herein may comprise 13 steps. By way of non-limiting example, the steps for IDQ may include:

1. Sample entry
2. Sample moves to lysis chamber
3. Cell lysis
4. Sample moves to neutralization region

### 5. Neutralization

6. Sample moves to hybridization/detection chamber

### 7. Hybridization

8. Sample moves to waste chamber
9. First wash valve is opened
10. First wash flows through hybridization/detection chamber
11. Second wash valve is opened
12. Second wash flows through hybridization/detection chamber

### 13. Detection

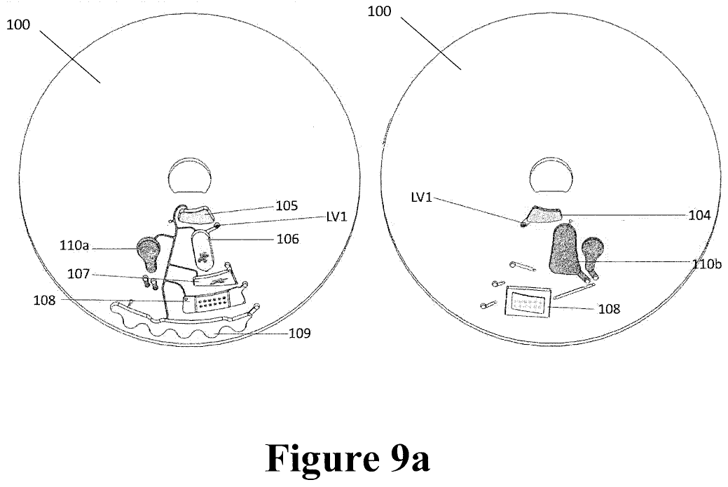

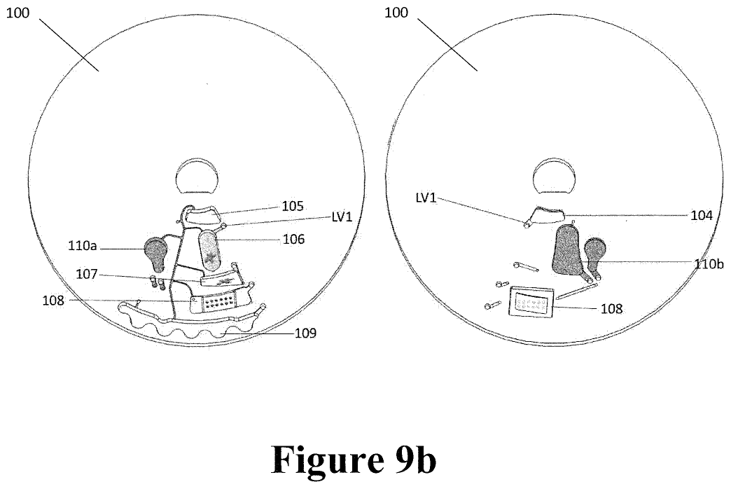

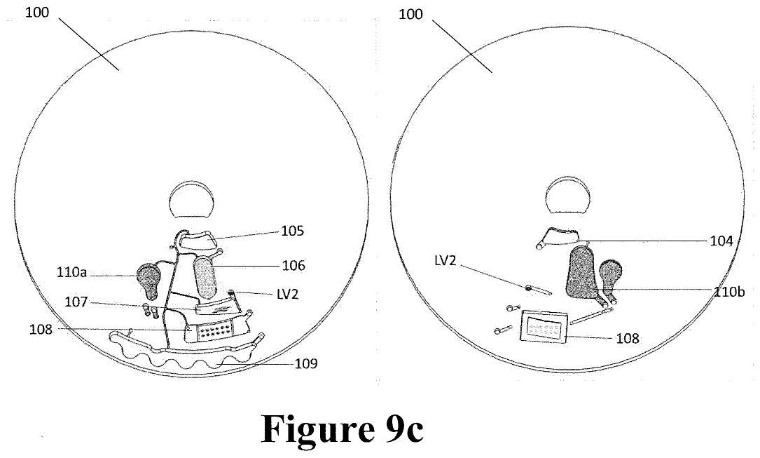

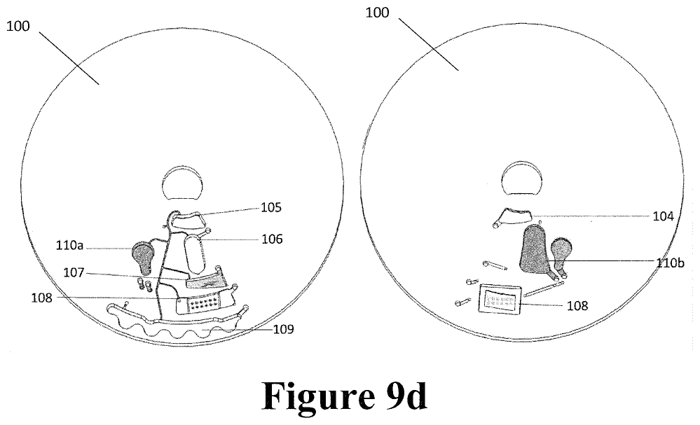

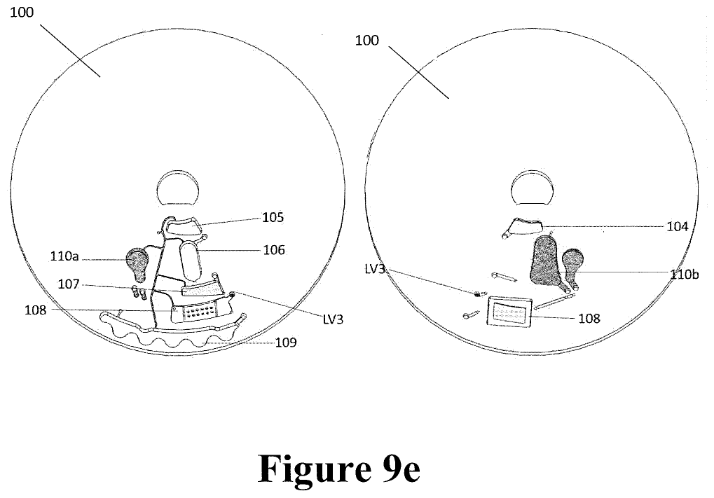

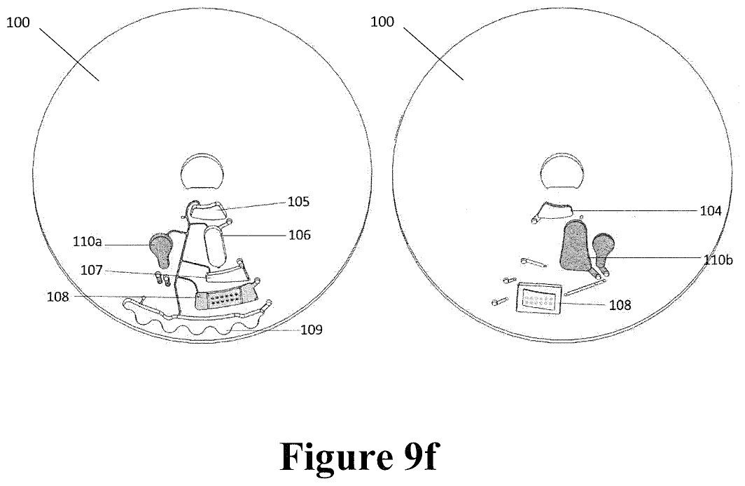

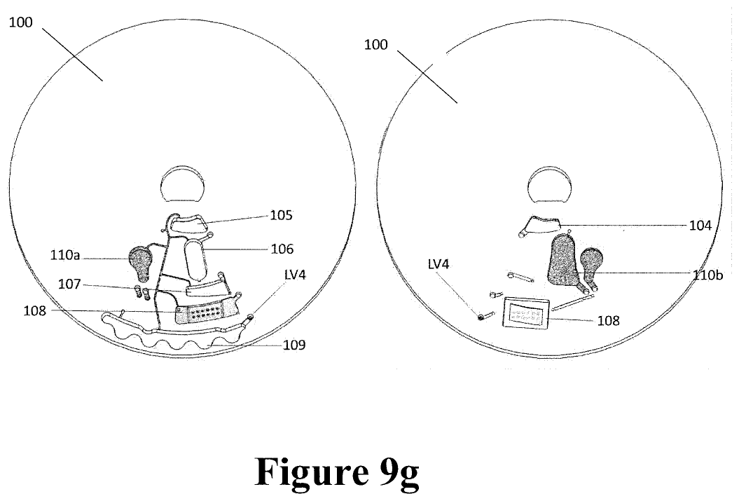

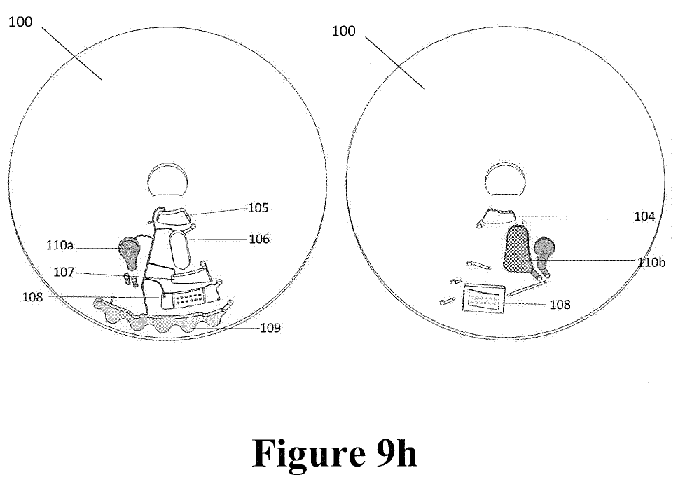

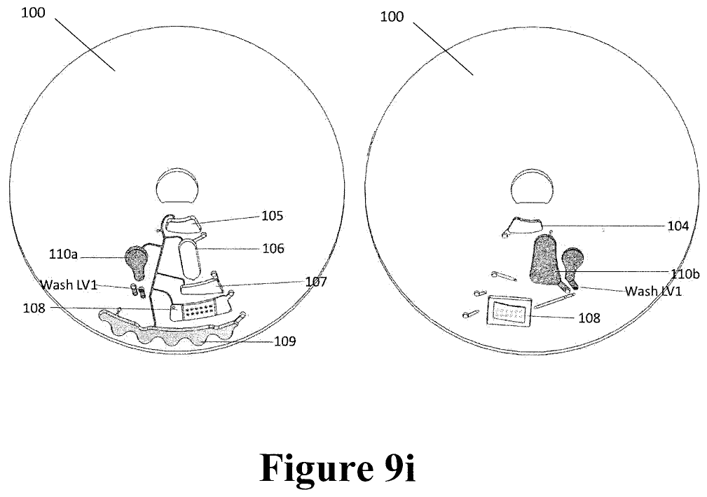

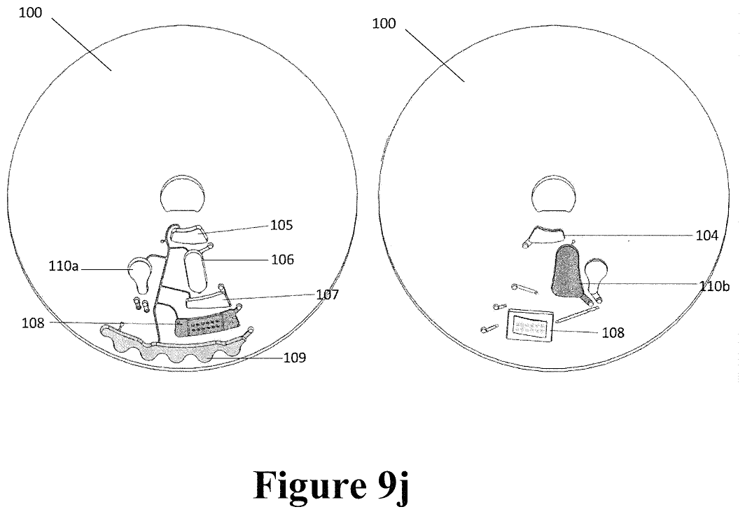

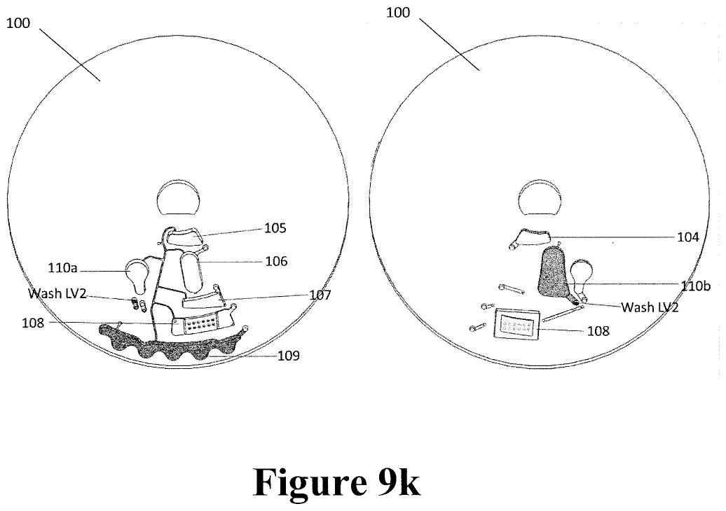

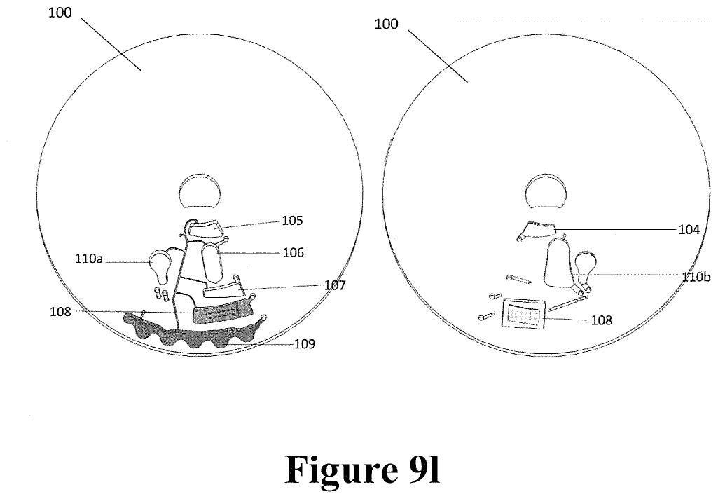

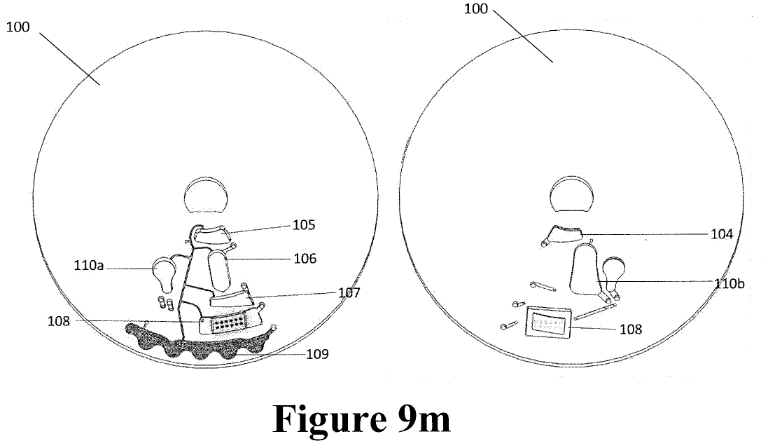

FIGS. 9a-m provide a pictorial representation of each step in a particular embodiment of the process of performing IDQ on a clinical specimen on a microfluidic cartridge 100 as disclosed herein. A representative table indicating the protocol and timing for each step in the IDQ process is included with FIGS. 9a-m.

As shown in FIG. 9a, step 1 in the IDQ process may comprise pipetting a clinical specimen into a sample entry point or fluid inlet region (see, e.g., FIG. 1b, 103). In certain embodiments, the time required to pipette in the clinical specimen may be about around 1 minute. After the sample is pipetted into the sample entry point, the valve in the microfluidic channel between the sample entry point and cell lysis chamber (referred to as LV1 in FIG. 9a) may be opened. In certain embodiments, the time required to open LV1 may be about around 1 minute.

As shown in FIG. 9b, step 2 in the IDQ process may comprise spinning the microfluidic cartridge 100 in order to move the clinical specimen from sample entry point or fluid inlet region to a cell lysis chamber (see, e.g., 106 FIG. 1a). In certain embodiments, step 2 may be carried out at a speed of about around 4000 RPM. In certain embodiments, the time required to move the clinical specimen from sample entry point or fluid inlet region to the cell lysis chamber may be about around 1 minute.

As shown in FIG. 9c, step 3 in the IDQ process may comprise lysing the clinical specimen within the cell lysis chamber. In certain embodiments, the lysis of step 3 may be carried out by spinning the microfluidic cartridge 100 at a speed of about around 200 RPM. In certain embodiments, the time required to lyse the clinical specimen may be around about 5 minutes. As shown in FIG. 9c, step 3 in the IDQ process may further comprise positioning a laser (or other external actuator) and then firing the laser (or operating the external actuator) to open the valve in the in the microfluidic channel between the cell lysis chamber and the neutralization region (referred to as LV 2 in FIG. 9c). In certain embodiments, the time required to open LV2 may be about around 1 minute.

As shown in FIG. 9d, step 4 in the IDQ process may comprise spinning the microfluidic cartridge 100 in order to move the clinical specimen from the cell lysis chamber to a neutralization region (see, e.g., 107, FIG. 1a). In certain embodiments, step 4 may be carried out at a speed of about around 4000 RPM. In certain embodiments, the time required to move the clinical specimen from the cell lysis chamber to the neutralization region may be about around 1 minute.

As shown in FIG. 9e, step 5 in the IDQ process may comprise neutralizing the clinical specimen within the neutralization region. In certain embodiments, the neutralization of step 5 may be carried out by oscillating the microfluidic cartridge 100 at an angle of around about 180 degrees with a frequency of around about 4 Hz. In certain embodiments, the time required to neutralize the clinical specimen may be around about 1 minute. As shown in FIG. 9e, step 5 in the IDQ process may further comprise positioning a laser (or other external actuator) and then firing the laser (or operating the external actuator) to open the valve in the in the microfluidic channel between the neutralization region and a hybridization/detection chamber (referred to as LV 3 in FIG. 9e). In certain embodiments, the time required to open LV3 may be about around 1 minute.

As shown in FIG. 9f, step 6 in the IDQ process may comprise spinning the microfluidic cartridge 100 in order to move the clinical specimen from the neutralization region to a hybridization/detection chamber (see, e.g., 108, FIG. 1a). In certain embodiments, step 6 may be carried out at a speed of about around 4000 RPM. In certain embodiments, the time required to move the clinical specimen from the neutralization region to the hybridization/detection chamber may be about around 1 minute.

As shown in FIG. 9g, step 7 in the IDQ process may comprise hybridizing the clinical specimen within the hybridization/detection chamber. In certain embodiments, the hybridization of step 7 may be carried out by oscillating the microfluidic cartridge 100 at an angle of around about 180 degrees with a frequency of around about 4 Hz. In certain embodiments, the time required to hybridize the clinical specimen may be around about 30 minutes. As shown in FIG. 9g, step 7 in the IDQ process may further comprise positioning a laser (or other external actuator) and then firing the laser (or operating the external actuator) to open the valve in the in the microfluidic channel between the hybridization/detection chamber and a waste chamber (referred to as LV 4 in FIG. 9g). In certain embodiments, the time required to open LV4 may be about around 1 minute.

As shown in FIG. 9h, step 8 in the IDQ process may comprise spinning the microfluidic cartridge 100 in order to move the clinical specimen from the hybridization/detection chamber to a waste chamber (see, e.g., 109 FIG. 1a). In certain embodiments, step 8 may be carried out at a speed of about around 4000 RPM. In certain embodiments, the time required to move the clinical specimen from the hybridization/detection chamber to the waste chamber may be about around 1 minute.

As shown in FIG. 9i, step 9 in the IDQ process may comprise positioning a laser (or other external actuator) and then firing the laser (or operating the external actuator) to open the valve in the in the microfluidic channel between the hybridization/detection chamber and a first wash chamber (referred to as Wash LV 1 in FIG. 9i). In certain embodiments, the time required to open Wash LV1 may be about around 1 minute.

As shown in FIG. 9j, step 10 in the IDQ process may comprise two stages. The first stage (wash stage) may comprise spinning the microfluidic cartridge 100 in order to move a wash buffer from a first wash chamber (see, e.g., 110a FIGS. 1a/b) to the hybridization/detection chamber. In certain embodiments, the wash stage of step 10 may be carried out at a speed of about around 1000 RPM. In certain embodiments, the time required to move the wash buffer from the first wash chamber to the hybridization/detection chamber may be about around 1 minute. The second stage (spin dry stage) may comprise spinning the microfluidic cartridge 100 in order to dry out the hybridization/detection chamber. In certain embodiments, the spin dry stage of step 10 may be carried out at a speed of about around 4000 RPM. In certain embodiments, the time required to spin dry the hybridization/detection chamber may be about around 1 minute.

As shown in FIG. 9k, step 11 in the IDQ process may comprise positioning a laser (or other external actuator) and then firing the laser (or operating the external actuator) to open the valve in the in the microfluidic channel between the hybridization/detection chamber and a second wash chamber (referred to as Wash LV 2 in FIG. 9k). In certain embodiments, the time required to open Wash LV2 may be about around 1 minute.

As shown in FIG. 9l, step 12 in the IDQ process may comprise two stages. The first stage (wash stage) may comprise spinning the microfluidic cartridge 100 in order to move a wash buffer from a second wash chamber (see, e.g., 110b of FIG. 1b) to the hybridization/detection chamber. In certain embodiments, the wash stage of step 12 may be carried out at a speed of about around 1000 RPM. In certain embodiments, the time required to move the wash buffer from the second wash chamber to the hybridization/detection chamber may be about around 1 minute. The second stage (spin dry stage) may comprise spinning the microfluidic cartridge 100 in order to dry out the hybridization/detection chamber. In certain embodiments, the spin dry stage of step 12 may be carried out at a speed of about around 4000 RPM. In certain embodiments, the time required to spin dry the hybridization/detection chamber may be about around 1 minute.

As shown in FIG. 9m, step 13 in the IDQ process may comprise positioning a camera over the hybridization/detection chamber and detecting the remaining sample on the capture probes within the hybridization/detection chamber. In certain embodiments, the time required to carry out the detection step may be about around 1 minute.

### AST

In yet another of its aspects, the invention herein discloses a method of performing antimicrobial susceptibility testing (AST) on a clinical specimen, using the system described herein (see, e.g. FIG. 7b). As shown in FIG. 7b, the method for performing AST may comprise the steps of sample entry, incubation, cell lysis, neutralization, hybridization, and detection.

In certain preferred embodiments, the method of performing may involve the use of a microfluidic cartridge 100, as shown for example in FIGS. 1a and 1b and the use of a driving apparatus, as shown for example in FIGS. 2a-c. In certain preferred embodiments, the method of performing AST may comprise the steps of:
(a) dispensing a predetermined amount of an antibiotic 702 in the incubation chamber 106 of a microfluidic cartridge;
(b) securing the microfluidic cartridge 100 with respect to the driving apparatus 200;
(c) dispensing a precise volume of the clinical specimen 701 into the fluid inlet port 103;
(d) using the rotating motor system 203 of the driving apparatus 200 to rotate the microfluidic cartridge 100 at a predetermined speed to allow the clinical specimen within the fluid inlet port 103 to flow into the incubation chamber 104;
(e) using the rotating motor system 203 of the driving apparatus 200 to oscillate the microfluidic cartridge 100 at a predetermined oscillation frequency and angle to allow turbulent mixing of the clinical specimen 701 with the antibiotic 702 within the incubation chamber 105;
(f) incubating the incubation chamber 105;
(g) using the actuator 205 to open the valve 113a between the incubation chamber 105 and the lysis chamber 106;
(h) using the rotating motor system 203 of the driving apparatus 200 to rotate the microfluidic cartridge 100 at a predetermined speed to allow the clinical specimen within the incubation chamber 105 to flow into the lysis chamber 106;
(i) using the rotating motor system 203 of the driving apparatus 200 to rotate the microfluidic cartridge 100 at a predetermined speed and angle such that the array of permanent magnets 204 on the driving apparatus 200 interact with the magnetic lysis puck 401 and the chemical lysing agent 402 in the lysis chamber 106, generating sheer force to break open the cell walls of the microorganisms and chemically degrade the cells and their biological material contained in the clinical specimen and creating a lysate;
(j) using the actuator 205 to open the valve 113b between the lysis chamber 106 and the neutralization region 107;
(k) using the rotating motor system 203 of the driving apparatus 200 to rotate the microfluidic cartridge 100 at a predetermined speed to allow the clinical specimen lysate within the lysis chamber 106 to flow into the neutralization region 107;
(l) using the rotating motor system 203 of the driving apparatus 200 to oscillate the microfluidic cartridge 100 at a predetermined oscillation frequency and angle to allow the buffer solution 404 and a series of detector probes functionalized with fluorescent signaling molecules 405 within the neutralization region 107 to neutralize the clinical specimen lysate, creating neutralized lysate;
(m) using the actuator 205 to open the valve 113c between the neutralization region 107 and the detection/hybridization chamber 108;
(n) using the rotating motor system 203 of the driving apparatus 200 to rotate the microfluidic cartridge 100 at a predetermined speed to allow the neutralized lysate within the neutralization region 107 flow into the detection/hybridization chamber 108;
(o) using the rotating motor system 203 of the driving apparatus 200 to oscillate the microfluidic cartridge 100, allowing the neutralized lysate to hybridize and bind with complementary capture probes 406 within the detection/hybridization chamber 108, wherein the capture probes 406 are bound to the surface of a solid surface insert (e.g., plastic);
(p) using the actuator 205 to open the valve 113d between the detection/hybridization chamber 108 and the waste chamber 109;
(q) using the rotating motor system 203 of the driving apparatus 200 to rotate the microfluidic cartridge 100 at a predetermined speed to allow the biological fluid in the detection/hybridization chamber 108 to flow from the detection/hybridization chamber 108 into the waste chamber 109;
(r) using the actuator 205 to open the valve 113e between the wash chamber 110a and the detection/hybridization chamber 108;
(s) using the rotating motor system 203 of the driving apparatus 200 to rotate the microfluidic cartridge 100 at a predetermined speed to allow the wash buffer from the wash chamber 110a to flow from wash chamber 110a and into the detection/hybridization chamber 108;
(t) using the rotating motor system 203 of the driving apparatus 200 to position the detection/hybridization chamber 108 above the fluorescent detection system in the driving apparatus 200;
(u) imaging the biological fluid sample using a fluorescent microscope;
(v) using the computer configured with image processing software, to convert the outputting light intensity information from the fluorescent detection system to determine the density of bacteria within the clinical specimen; and
(w) using the computer configured with image processing software to test the antibiotic susceptibility of the clinical specimen.

By way of non-limiting example, the antibiotic susceptibility of a clinical specimen may be tested by comparing the bacterial density of a clinical specimen without antibiotic to the bacterial density of a clinical specimen that has been mixed with an antibiotic.

In certain embodiments, the method may further comprise the steps of:

In certain embodiments, washing may be done at about 1000 RPM to allow for a slow flow through of the wash buffer. By way of non-limiting example, in certain embodiments, the buffer from the first wash chamber 110a may completely fill the detection/hybridization chamber 108, then the buffer will slowly drain out of the detection/hybridization chamber 108 into the waste chamber. This process may then be repeated for the buffer from the second wash chamber 110b.

In certain embodiments, the process for antimicrobial susceptibility testing (AST) on a microfluidic cartridge such as those described herein may comprise 14 steps. By way of non-limiting example, the steps for AST may include:

1. Sample entry/dilution

2. Incubation

3. Sample moves to lysis chamber

4. Cell lysis

5. Sample moves to neutralization region

6. Neutralization

7. Sample moves to hybridization/detection chamber

8. Hybridization

9. Sample moves to waste chamber

10. First wash valve is opened

11. First wash flows through hybridization/detection chamber

12. Second wash valve is opened

13. Second wash flows through hybridization/detection chamber

14. Detection

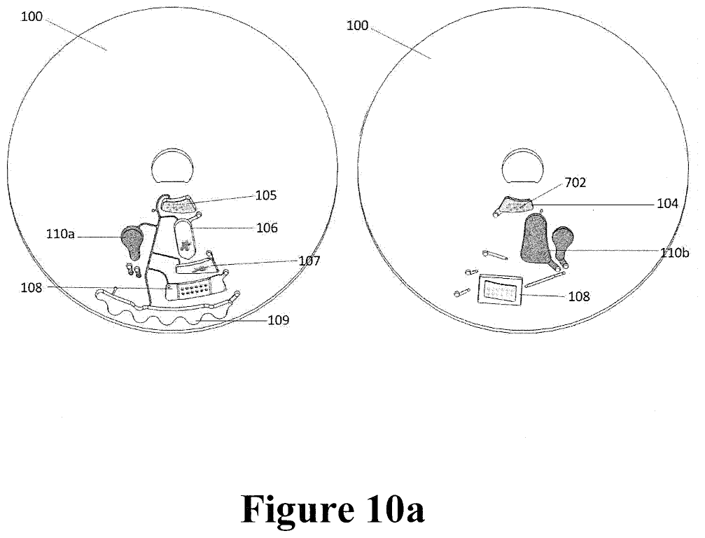

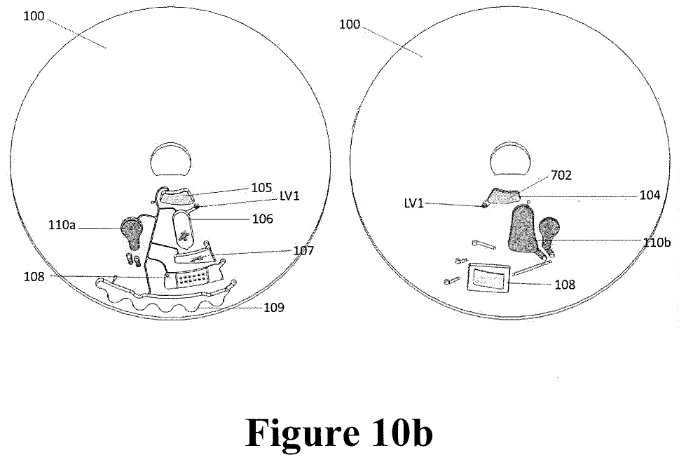

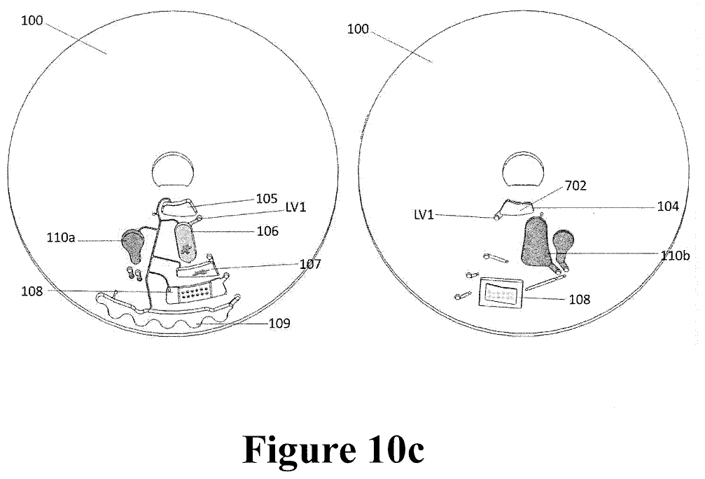

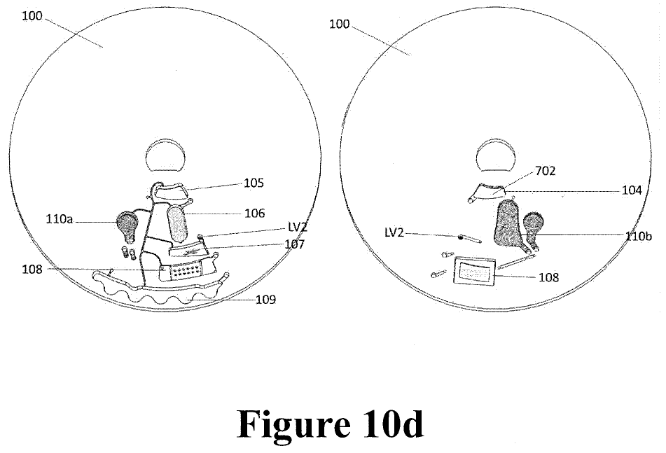

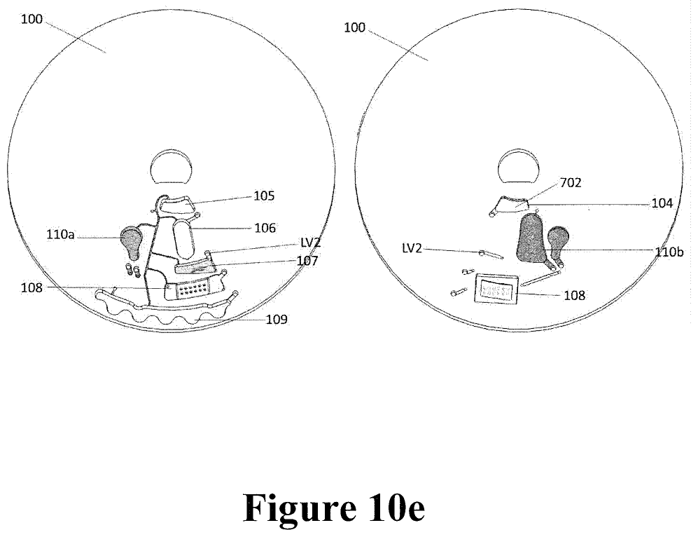

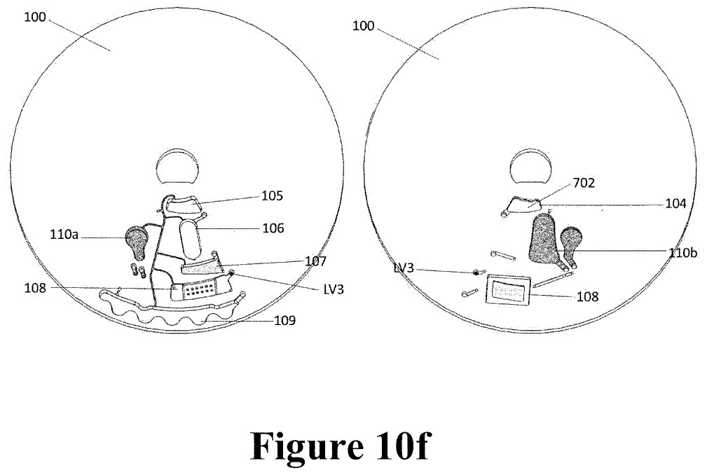

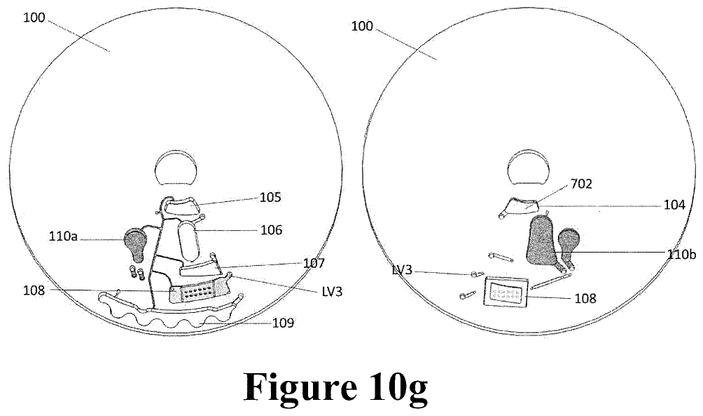

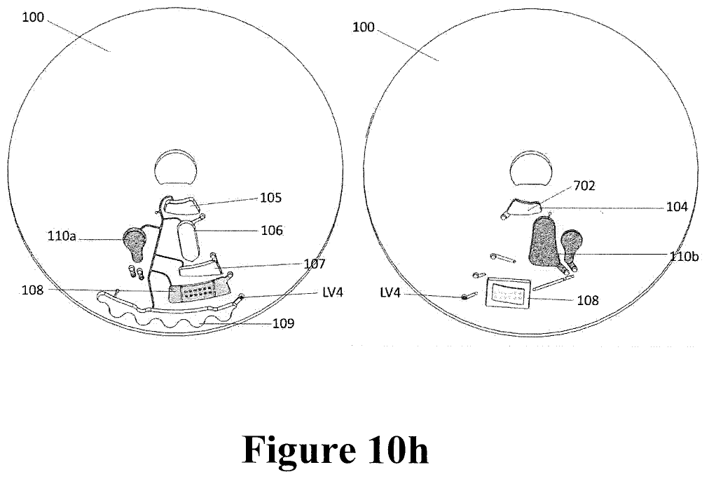

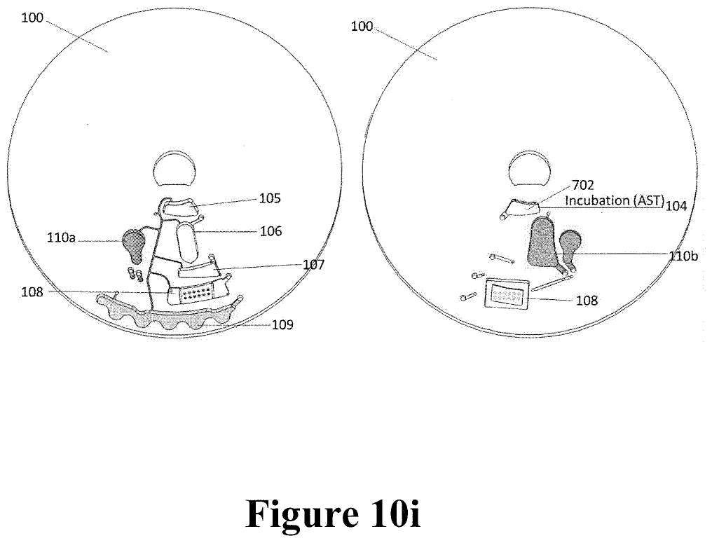

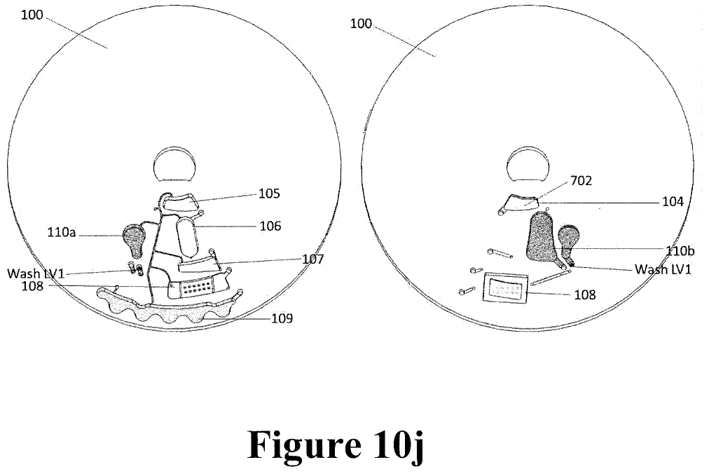

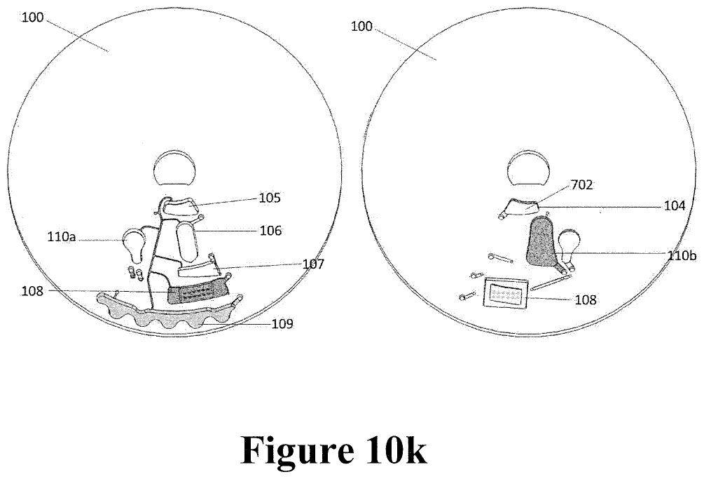

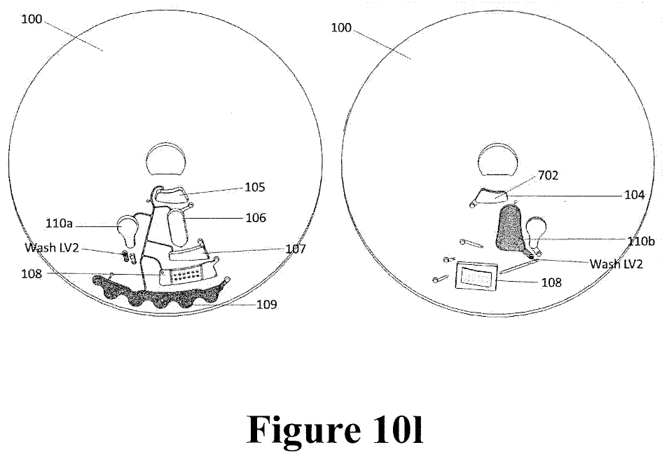

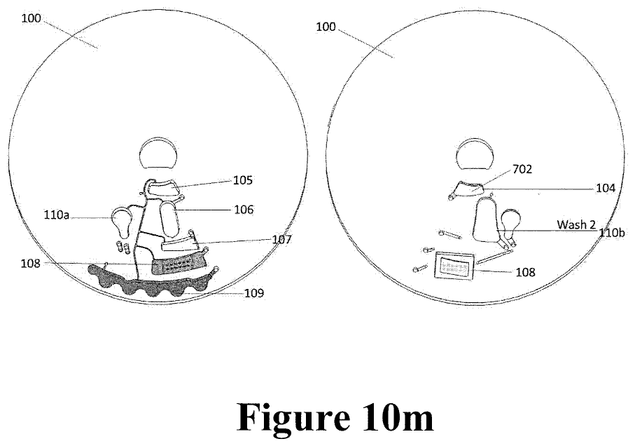

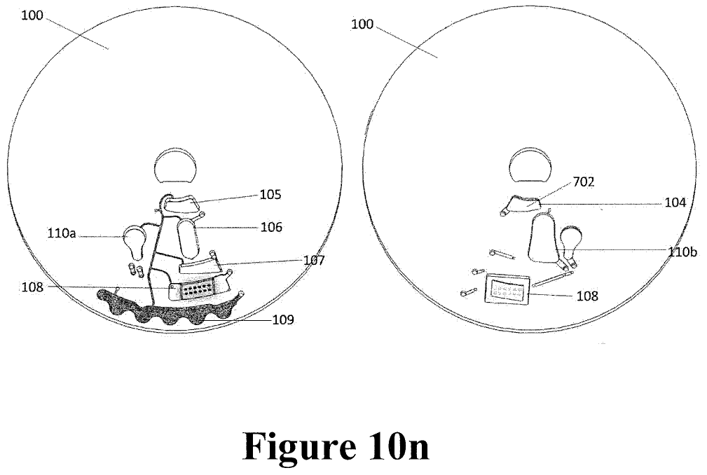

FIGS. 10a-n provide a pictorial representation of each step in a particular embodiment of the process of performing AST on a clinical specimen on a microfluidic cartridge 100 as disclosed herein. A representative table indicating the protocol and timing for each step in the AST process is included with FIGS. 10a-n.

As a starting point, before any steps of the AST process are carried out, a sample of antibiotic may be dispensed into an incubation chamber (see, e.g., 105, FIG. 1a) of a microfluidic cartridge 100.

As shown in FIG. 10a, step 1 in the AST process may comprise diluting and pipetting a clinical specimen into a sample entry point or fluid inlet region (see, e.g., FIG. 1b, 103). By way of non-limiting example, the clinical specimen may be diluted using a 1:10 sample dilution. In other examples, the clinical specimen may be diluted using a 1:100 sample dilution. In certain embodiments, the time required to dilute the clinical specimen may be about around 1 minute.

As shown in FIG. 10b, step 2 in the AST process may comprise incubating the clinical specimen in an incubation chamber (see, e.g., 105, FIG. 1a). In certain embodiments, the incubation of step 2 may be carried out by oscillating the microfluidic cartridge 100 at an angle of around about 180 degrees with a frequency of around about 1 Hz to 8 Hz, more preferably around about 4 Hz. In certain embodiments, the time required to incubate the clinical specimen may be from around 1 minute to around 10 hours, more preferably around about 90 minutes. In certain embodiments, the incubation step of the AST process may be carried out at a temperature in the range of about range of about 25° C. to 45° C., more preferably about around 37° C. As shown in FIG. 10b, step 2 in the AST process may further comprise positioning a laser (or other external actuator) and then firing the laser (or operating the external actuator) to open the valve in the in the microfluidic channel between the incubation chamber and a cell lysis chamber (referred to as LV 1 in FIG. 10b). In certain embodiments, the time required to open LV1 may be about around 1 minute.

As shown in FIG. 10c, step 3 in the AST process may comprise spinning the microfluidic cartridge 100 in order to move the clinical specimen from the incubation chamber to a cell lysis chamber (see, e.g., 106 FIG. 1a). In certain embodiments, step 3 may be carried out at a speed of about around 4000 RPM. In certain embodiments, the time required to move the clinical specimen from the incubation chamber to the cell lysis chamber may be about around 1 minute.

As shown in FIG. 10d, step 4 in the AST process may comprise lysing the clinical specimen within the cell lysis chamber. In certain embodiments, the lysis of step 4 may be carried out by spinning the microfluidic cartridge 100 at a speed of about around 200 RPM. In certain embodiments, the time required to lyse the clinical specimen may be around about 5 minutes. As shown in FIG. 10d, step 4 in the AST process may further comprise positioning a laser (or other external actuator) and then firing the laser (or operating the external actuator) to open the valve in the in the microfluidic channel between the cell lysis chamber and the neutralization region (referred to as LV 2 in FIG. 10d). In certain embodiments, the time required to open LV2 may be about around 1 minute.

As shown in FIG. 10e, step 5 in the AST process may comprise spinning the microfluidic cartridge 100 in order to move the clinical specimen from the cell lysis chamber to a neutralization region (see, e.g., 107, FIG. 1a). In certain embodiments, step 5 may be carried out at a speed of about around 4000 RPM. In certain embodiments, the time required to move the clinical specimen from the cell lysis chamber to the neutralization region may be about around 1 minute.

As shown in FIG. 10f, step 6 in the AST process may comprise neutralizing the clinical specimen within the neutralization region. In certain embodiments, the neutralization of step 6 may be carried out by oscillating the microfluidic cartridge 100 at an angle of around about 180 degrees with a frequency of around about 4 Hz. In certain embodiments, the time required to neutralize the clinical specimen may be around about 1 minute. As shown in FIG. 10f, step 6 in the AST process may further comprise positioning a laser (or other external actuator) and then firing the laser (or operating the external actuator) to open the valve in the in the microfluidic channel between the neutralization region and a hybridization/detection chamber (referred to as LV 3 in FIG. 10f). In certain embodiments, the time required to open LV3 may be about around 1 minute.

As shown in FIG. 10g, step 7 in the AST process may comprise spinning the microfluidic cartridge 100 in order to move the clinical specimen from the neutralization region to a hybridization/detection chamber (see, e.g., 108, FIG. 1a). In certain embodiments, step 7 may be carried out at a speed of about around 4000 RPM. In certain embodiments, the time required to move the clinical specimen from the neutralization region to the hybridization/detection chamber may be about around 1 minute.

As shown in FIG. 10h, step 8 in the AST process may comprise hybridizing the clinical specimen within the hybridization/detection chamber. In certain embodiments, the hybridization of step 8 may be carried out by oscillating the microfluidic cartridge 100 at an angle of around about 180 degrees with a frequency of around about 4 Hz. In certain embodiments, the time required to hybridize the clinical specimen may be around about 30 minutes. As shown in FIG. 10h, step 8 in the AST process may further comprise positioning a laser (or other external actuator) and then firing the laser (or operating the external actuator) to open the valve in the in the microfluidic channel between the hybridization/detection chamber and a waste chamber (referred to as LV 4 in FIG. 10h). In certain embodiments, the time required to open LV4 may be about around 1 minute.

As shown in FIG. 10i, step 9 in the AST process may comprise spinning the microfluidic cartridge 100 in order to move the clinical specimen from the hybridization/detection chamber to a waste chamber (see, e.g., 109 FIG. 1a). In certain embodiments, step 9 may be carried out at a speed of about around 4000 RPM. In certain embodiments, the time required to move the clinical specimen from the hybridization/detection chamber to the waste chamber may be about around 1 minute.

As shown in FIG. 10j, step 10 in the AST process may comprise positioning a laser (or other external actuator) and then firing the laser (or operating the external actuator) to open the valve in the in the microfluidic channel between the hybridization/detection chamber and a first wash chamber (referred to as Wash LV 1 in FIG. 10j). In certain embodiments, the time required to open Wash LV1 may be about around 1 minute.

As shown in FIG. 10k, step 11 in the AST process may comprise two stages. The first stage (wash stage) may comprise spinning the microfluidic cartridge 100 in order to move a wash buffer from a first wash chamber (see, e.g., 110a FIGS. 1a/b) to the hybridization/detection chamber. In certain embodiments, the wash stage of step 11 may be carried out at a speed of about around 1000 RPM. In certain embodiments, the time required to move the wash buffer from the first wash chamber to the hybridization/detection chamber may be about around 1 minute. The second stage (spin dry stage) may comprise spinning the microfluidic cartridge 100 in order to dry out the hybridization/detection chamber. In certain embodiments, the spin dry stage of step 11 may be carried out at a speed of about around 4000 RPM. In certain embodiments, the time required to spin dry the hybridization/detection chamber may be about around 1 minute.

As shown in FIG. 10l, step 12 in the AST process may comprise positioning a laser (or other external actuator) and then firing the laser (or operating the external actuator) to open the valve in the in the microfluidic channel between the hybridization/detection chamber and a second wash chamber (referred to as Wash LV 2 in FIG. 10l). In certain embodiments, the time required to open Wash LV2 may be about around 1 minute.

As shown in FIG. 10m, step 13 in the AST process may comprise two stages. The first stage (wash stage) may comprise spinning the microfluidic cartridge 100 in order to move a wash buffer from a second wash chamber (see, e.g., 110b FIG. 1b) to the hybridization/detection chamber. In certain embodiments, the wash stage of step 13 may be carried out at a speed of about around 1000 RPM. In certain embodiments, the time required to move the wash buffer from the second wash chamber to the hybridization/detection chamber may be about around 1 minute. The second stage (spin dry stage) may comprise spinning the microfluidic cartridge 100 in order to dry out the hybridization/detection chamber. In certain embodiments, the spin dry stage of step 13 may be carried out at a speed of about around 4000 RPM. In certain embodiments, the time required to spin dry the hybridization/detection chamber may be about around 1 minute.

As shown in FIG. 10n, step 14 in the AST process may comprise positioning a camera over the hybridization/detection chamber and detecting the remaining sample on the capture probes within the hybridization/detection chamber. In certain embodiments, the time required to carry out the detection step may be about around 1 minute.

In certain embodiments, the minimum spin speed of the rotational apparatus may be about 1000 RPM and the maximum spin speed may be about 10,000 RPM.

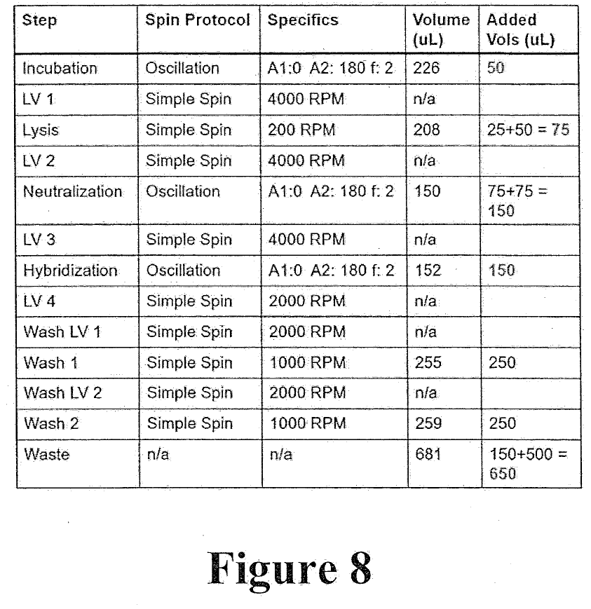

FIG. 8 provides an example of the representative steps performed for an example AST process.

The table in FIG. 8 provides each step in the process, including the spin protocol for each step. LV1, LV2, LV3 and LV4 refer to the valve between the incubation and lysis chambers (LV1), the valve between the lysis chamber and neutralization region (LV2), the valve between the neutralization region and detection/hybridization chamber (LV3) and the valve between the detection/hybridization chamber and the waste chamber (LV4). Wash LV1 and Wash LV2 refer to the valves between wash chambers 1 and 2, respectively, and the detection/hybridization chamber. RPM, as used in FIG. 8, stands for “rotations per minute”. This is the speed input for the spinning step. For the oscillation step, A1 is an angular position, A2 is an angular position, and f is an oscillation frequency. In certain embodiments, an IDQ assay may involve the same steps as those shown in FIG. 8, using the same parameters, with the exception of the incubation step.

The disclosure illustratively described herein can suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

1. A rotatable microfluidic cartridge for processing and analyzing one or more specimens comprising:
a body portion having a first surface and an opposing second surface; a fluid inlet region configured to receive the specimen; and at least a first microfluidic flow path extending downstream from the fluid inlet port through the body of the cartridge, wherein the cartridge is configured to be secured with respect to a driving apparatus and configured to be rotated about a rotational axis when the driving apparatus is in operation, and wherein the first microfluidic flow path comprises:
(a) an incubation chamber;
(b) a lysis chamber;
(c) a neutralization region; and
(d) a detection/hybridization chamber;
wherein (a), (b), (c), and (d) are all connected through a series of microfluidic channels that are configured to allow the specimen to flow from (a) to (b) to (c) to (d) when the cartridge is rotated or oscillated.

2. The apparatus disclosed in claim 1, wherein the lysis chamber comprises at least one magnetic lysis puck that can translate within the chamber to cause disruption of a cellular membrane in the specimen when the cartridge is rotated, and wherein the lysis chamber contains at least one chemical lysing agent.

3. The apparatus disclosed in claim 1, wherein the incubation chamber is configured to allow turbulent mixing within the chamber when the cartridge oscillated back and forth along an oscillation path at a predetermined oscillation frequency.

4. The apparatus disclosed in claim 1, wherein the neutralization region comprises a buffer solution capable of neutralizing the specimen and a series of detector probes that are functionalized with fluorescent signaling molecules.

5. The apparatus disclosed in claim 1, wherein the detection/hybridization chamber comprises a series of capture probes, wherein the capture probes are immobilized on a plastic surface such that the neutralized specimen is allowed to hybridize and bind with the capture probes when the microfluidic cartridge is oscillated back and forth along an oscillation path at a predetermined oscillation frequency.

6. The apparatus disclosed in any of claims 1 to 5, wherein the microfluidic flow path further comprises at least one valve, the valve being actuatable between a closed configuration in which the microfluidic flow path is blocked and an open position to allow the movement of the specimen within the microfluidic flow path, and wherein the valve is be actuatable by an actuator that is external the body portion.

7. The apparatus of claim 6, wherein the microfluidic flow path comprises a valve between the incubation chamber and the lysis chamber.

8. The apparatus of claim 6, wherein the microfluidic flow path comprises a valve between the lysis chamber and the neutralization region.

9. The apparatus of claim 6, wherein the microfluidic flow path comprises a valve between the neutralization region and the detection/hybridization chamber.

10. The apparatus of claim 6, wherein the microfluidic flow path comprises a valve between the incubation chamber and the lysis chamber and a valve between the lysis chamber and the neutralization region and a valve between the neutralization region and the detection/hybridization chamber.

11. The apparatus disclosed in any of claims 1 to 10, wherein the microfluidic flow path further comprises a waste chamber connected to and located downstream from the detection/hybridization chamber, wherein the waste chamber is configured to receive the specimen from the detection/hybridization chamber when the cartridge is rotated or oscillated.

12. The apparatus of claim 11, wherein the microfluidic flow path comprises a valve between the waste chamber and the detection/hybridization chamber.

13. The apparatus disclosed in any of claims 1 through 12, wherein the microfluidic flow path further comprises at least one wash chamber.

14. The apparatus disclosed in claim 13, wherein the at least one wash chamber contains a wash buffer.

15. The apparatus disclosed in claim 14, wherein the detection/hybridization chamber is connected to and located downstream and the wash chamber, wherein the detection/hybridization chamber is configured to receive the wash buffer from the wash chamber when the cartridge is rotated or oscillated.

16. The apparatus of claim 15, wherein the microfluidic flow path comprises a valve between the wash chamber and the detection/hybridization chamber.

17. The apparatus disclosed in any of claims 6 through 16, where in each valve comprises:
(a) a first blocking member positioned in the microfluidic flow path between two sections of said flow path and fluidly isolating the second section of the microfluidic flow path from the first section, the first blocking member being spaced inwardly from the first surface and the second surface of the body portion of the microfluidic cartridge;
wherein the first blocking member is configured to be at least partially destroyable via the actuator that is external to the body portion when the cartridge is in use, thereby establishing fluid communication between the first section and the second section of the microfluidic flow path and allowing the clinical specimen to flow from the first section to the second section.

18. The apparatus disclosed in claim 17, wherein the first blocking member is meltable by a laser without melting the adjacent portions of the body.

19. The apparatus disclosed in claim 17 or 18, wherein the actuator comprises at least one of a laser and an ultrasound transducer.

20. The apparatus disclosed in claim 19, wherein the actuator comprises a laser and a first transmission portion of the first surface is disposed between the first blocking member and the laser so when the apparatus is in use the laser beam passes through the first transmission portion before reaching the blocking member, and the first transmission portion of the first surface remains intact.

21. A rotatable microfluidic cartridge for processing and analyzing one or more specimens, the cartridge comprising:
a body portion having a first surface and an opposing second surface;
a fluid inlet region configured to receive the specimen; and
at least a first microfluidic flow path extending downstream from the fluid inlet port through the body of the cartridge, wherein the cartridge is configured to be secured with respect to a driving apparatus and configured to be rotated about a rotational axis when the driving apparatus is in operation, and wherein the first microfluidic flow path comprises:
(a) an incubation chamber located downstream from the fluid inlet region, wherein the incubation chamber is configured to receive the specimen from the fluid inlet region when the cartridge is rotated;
(b) a lysis chamber connected to and located downstream from the incubation chamber, wherein the lysis chamber is configured to receive the specimen from the incubation chamber when the cartridge is rotated;
(c) a neutralization region connected to and located downstream from the lysis chamber, wherein the neutralization region is configured to receive the specimen from the lysis chamber when the cartridge is rotated; and
(d) a detection/hybridization chamber connected to and located downstream from the neutralization region, wherein the detection/hybridization chamber is configured to receive the specimen from the neutralization region when the cartridge is rotated.

22. The apparatus disclosed in claim 21, wherein the microfluidic flow path further comprises a waste chamber connected to and located downstream from the detection/hybridization chamber, wherein the waste chamber is configured to receive the specimen from the detection/hybridization chamber when the cartridge is rotated.

23. The apparatus disclosed in claim 21 or 22, wherein the microfluidic flow path further comprises at least one wash chamber connected to and located upstream from the detection/hybridization chamber, wherein the wash chamber comprises a wash buffer, and wherein the detection/hybridization chamber is configured to receive the wash buffer from the wash chamber when the cartridge is rotated.

24. The apparatus disclosed in any one of claims 21 through 23, wherein the microfluidic flow path further comprises at least one valve, the valve being actuatable between a closed configuration in which the microfluidic flow path is blocked and an open position to allow the movement of the specimen within the microfluidic flow path, and wherein the valve is be actuatable by an actuator that is external the body portion.

25. The apparatus of claim 24, wherein the microfluidic flow path comprises a valve between the incubation chamber and the lysis chamber.

26. The apparatus of claim 24, wherein the microfluidic flow path comprises a valve between the lysis chamber and the neutralization region.

27. The apparatus of claim 24, wherein the microfluidic flow path comprises a valve between the neutralization region and the detection/hybridization chamber.

28. The apparatus of claim 24, wherein the microfluidic flow path comprises a valve between the wash chamber and the detection/hybridization chamber.

29. The apparatus of claim 24, wherein the microfluidic flow path comprises a valve between the detection/hybridization chamber and the waste chamber.

30. The apparatus of claim 24, wherein the microfluidic flow path comprises a valve between the incubation chamber and the lysis chamber and a valve between the lysis chamber and the neutralization region and a valve between the neutralization region and the detection/hybridization chamber and a valve between the wash chamber and the detection/hybridization chamber and a valve between the detection/hybridization chamber and the waste chamber.

31. The apparatus disclosed in any of claims 21 through 30, wherein the microfluidic flow path further comprises a closed-loop ventilation network, wherein the ventilation network allows air to move around the microfluidic flow path without exposing the interior of the microfluidic flow path to the surrounding air.

32. The apparatus of claim 31, wherein the closed-loop ventilation network comprises:
(a) a first vent line which is connected to and located radially inward from the incubation chamber such that the clinical specimen within the incubation chamber cannot enter the vent line when the cartridge is spinning or oscillating;
(b) a second vent line which is connected to and located radially inward from the lysis chamber such that the specimen within the lysis chamber cannot enter the vent line when the cartridge is spinning or oscillating;
(c) a third vent line which is connected to and located radially inward from the neutralization region such that the specimen within the neutralization region cannot enter the vent line when the cartridge is spinning or oscillating; and
(d) a fourth vent line which is connected to and located radially inward from the detection/hybridization chamber such that the specimen within the detection/hybridization chamber cannot enter the vent line when the cartridge is spinning or oscillating;
wherein the first, second, third and fourth vent lines are in fluid communication with each other.

33. The apparatus disclosed in any of claims 24 through 32, where in each valve comprises:
a first blocking member positioned in the microfluidic flow path between two sections of said flow path and fluidly isolating the second section of the microfluidic flow path from the first section, the first blocking member being spaced inwardly from the first surface and the second surface of the body portion of the microfluidic cartridge;
wherein the first blocking member is configured to be at least partially destroyable via an actuator that is external to the body portion when the cartridge is in use, thereby establishing fluid communication between the first section and the second section of the microfluidic flow path and allowing the specimen to flow from the first section to the second section.

34. The apparatus disclosed in claim 33, wherein the first blocking member is meltable by a laser without melting the adjacent portions of the body.

35. The apparatus disclosed in claim 33 or 34, wherein the actuator comprises at least one of a laser and an ultrasound transducer.

36. The apparatus disclosed in any of claims 33-35, wherein the actuator comprises a laser and a first transmission portion of the first surface is disposed between the first blocking member and the laser so when the apparatus is in use the laser beam passes through the first transmission portion before reaching the blocking member, and the first transmission portion of the first surface remains intact.

37. The apparatus disclosed in any of claims 21-36, wherein the lysis chamber comprises at least one magnetic lysis puck that can translate within the chamber to cause disruption of a cellular membrane in the specimen when the cartridge is rotated, and wherein the lysis chamber contains at least one chemical lysing agent.

38. The apparatus disclosed in any of claims 21-36, wherein the lysis chamber contains a slurry of ceramic beads, glass beads, zirconium beads, silica-zirconium beads, steel beads or any combination of two or more of these or other chemically inert abrasive microparticles.

39. The apparatus disclosed in any of claims 21-38, wherein the incubation chamber is configured to allow turbulent mixing within the chamber when the cartridge oscillated back and forth along an oscillation path at a predetermined oscillation frequency.

40. The apparatus disclosed in any of claims 21-39, wherein the neutralization region comprises a buffer solution capable of neutralizing the specimen and a series of detector probes that are functionalized with fluorescent signaling molecules.

41. The apparatus disclosed in any of claims 21-40, wherein the detection/hybridization chamber comprises a series of capture probes, wherein the capture probes are immobilized on a plastic surface such that the neutralized clinical specimen is allowed to hybridize and bind with the capture probes when the microfluidic cartridge is oscillated back and forth along an oscillation path at a predetermined oscillation frequency.

42. A system for processing and analyzing one or more specimens comprising:
a driving apparatus comprising a rotatable motor, an array of permanent magnets, a laser and a fluorescent detection system, wherein the driving apparatus may be configured to receive a microfluidic cartridge and to rotate the cartridge;
a rotatable microfluidic cartridge for processing and analyzing one or more clinical specimens comprising a body portion having a first surface and an opposing second surface, a fluid inlet region configured to receive the specimen, and at least a first microfluidic flow path extending downstream from the fluid inlet port through the body of the cartridge, wherein the cartridge is configured to be secured with respect to a driving apparatus and configured to be rotated about a rotational axis when the driving apparatus is in operation, and wherein the first microfluidic flow path comprises:
(a) an incubation chamber;
(b) a lysis chamber;
(c) a neutralization region; and
(d) a detection/hybridization chamber;
wherein (a), (b), (c), and (d) are all connected through a series of microfluidic channels that are configured to allow the specimen to flow from (a) to (b) to (c) to (d) when the cartridge is rotated or oscillated.

43. The system of claim 42, wherein the microfluidic flow path further comprises at least one valve, the valve being actuatable between a closed configuration in which the microfluidic flow path is blocked and an open position to allow the movement of the specimen within the microfluidic flow path, and wherein the valve is be actuatable by an actuator that is external the body portion.

44. The system of claim 43, wherein the microfluidic flow path comprises a valve between the incubation chamber and the lysis chamber.

45. The system of claim 43, wherein the microfluidic flow path comprises a valve between the lysis chamber and the neutralization region.

46. The system of claim 43, wherein the microfluidic flow path comprises a valve between the neutralization region and the detection/hybridization chamber.

47. The system of claim 43, wherein the microfluidic flow path comprises a valve between the incubation chamber and the lysis chamber and a valve between the lysis chamber and the neutralization region and a valve between the neutralization region and the detection/hybridization chamber.

48. The system disclosed in any of claims 42 through 47, wherein the microfluidic flow path further comprises a waste chamber connected to and located downstream from the detection/hybridization chamber, wherein the waste chamber is configured to receive the specimen from the detection/hybridization chamber when the cartridge is rotated or oscillated.

49. The system of claim 48, wherein the microfluidic flow path comprises a valve between the waste chamber and the detection/hybridization chamber.

50. The system disclosed in any of claims 42 through 49, wherein the microfluidic flow path further comprises at least one wash chamber.

51. The system disclosed in claim 50, wherein the wash chamber contains a wash buffer.

52. The system disclosed in claim 51, wherein the detection/hybridization chamber is connected to and located downstream from the wash chamber, wherein the detection/hybridization chamber is configured to receive the wash buffer from the wash chamber when the cartridge is rotated or oscillated.

53. The system of claim 52, wherein the microfluidic flow path comprises a valve between the wash chamber and the detection/hybridization chamber.

54. The system disclosed in any of claims 42 through 53, wherein the microfluidic flow path further comprises a closed-loop ventilation network, wherein the ventilation network allows air to move around the microfluidic flow path without exposing the interior of the microfluidic flow path to the surrounding air.

55. The system disclosed in any of claims 42 through 54, wherein each valve comprises:
a first blocking member positioned in the microfluidic flow path between two sections of said flow path and fluidly isolating the second section of the microfluidic flow path from the first section, the first blocking member being spaced inwardly from the first surface and the second surface of the body portion of the microfluidic cartridge;
wherein the first blocking member is configured to be at least partially destroyable via the actuator that is external to the body portion when the cartridge is in use, thereby establishing fluid communication between the first section and the second section of the microfluidic flow path and allowing the specimen to flow from the first section to the second section.

56. The system disclosed in claim 55, wherein the first blocking member is meltable by a laser without melting the adjacent portions of the body.

57. The system disclosed in claim 55 or 56, wherein the actuator comprises at least one of a laser and an ultrasound transducer.

58. The system disclosed in any of claims 55 through 57, wherein the actuator comprises a laser and a first transmission portion of the first surface is disposed between the first blocking member and the laser so when the apparatus is in use the laser beam passes through the first transmission portion before reaching the blocking member, and the first transmission portion of the first surface remains intact.

59. The system disclosed in any of claims 42 through 58, further comprising a computer configured with image processing software, wherein the image processing software is capable of outputting light intensity information from the fluorescent detection system and wherein the computer is configured with an algorithm to use the light intensity information to determine the density of bacteria within the clinical specimen.

60. The system disclosed in any of claims 42 through 59, wherein the microfluidic cartridge is disposable.

61. The system disclosed in any of claims 42 through 60, wherein the microfluidic cartridge is configured to perform bacteria identification and quantification.

62. The system disclosed in any of claims 42 through 60, wherein the microfluidic cartridge is configured to perform antimicrobial susceptibility testing.

63. The system disclosed in any of claims 42 through 62, wherein the microfluidic cartridge is circle shaped.

64. The system disclosed in any of claims 42 through 62, wherein the microfluidic cartridge is wedge shaped.

65. The system disclosed in claim 64, wherein three or more wedge shaped cartridges are configured to fit into a circular carousel.

66. The system disclosed in any of claims 42 through 65, wherein the driving apparatus further comprises a temperature control system, wherein the temperature control system is configured to maintain a desired temperature within the driving apparatus.

67. The system disclosed in claim 66, wherein the temperature control system comprises a heater, at least one blower and a thermocouple.

68. The system disclosed in claim 66 or 67, wherein the desired temperature within the driving apparatus is in the range of about 25° C. to 45° C.

69. The system disclosed in claim 68, wherein the desired temperature within the driving apparatus is around about 37° C.

70. The system disclosed in any of claims 42 through 69, wherein the driving apparatus further comprises a safety switch, wherein the safety switch is configured to prevent an operator of the driving apparatus from being exposed to harmful lasers.

71. The system disclosed in any of claims 42 through 69, wherein the incubation chamber is located downstream from the fluid inlet region, and wherein the incubation chamber is configured to receive the specimen from the fluid inlet region when the cartridge is rotated.

72. The system disclosed in any of claims 42 through 71, wherein the lysis chamber is connected to and located downstream from the incubation chamber, and wherein the lysis chamber is configured to receive the specimen from the incubation chamber when the cartridge is rotated.

73. The system disclosed in any of claims 42 through 72, wherein the neutralization region connected to and located downstream from the lysis chamber, and wherein the neutralization region is configured to receive the specimen from the lysis chamber when the cartridge is rotated.

74. The system disclosed in any of claims 42 through 73, wherein the detection/hybridization chamber connected to and located downstream from the neutralization region, and wherein the detection/hybridization chamber is configured to receive the specimen from the neutralization region when the cartridge is rotated.

75. The system of any of claims 54 through 74, wherein the closed-loop ventilation network comprises:
(a) a first vent line which is connected to and located radially inward from the incubation chamber such that the specimen within the incubation chamber cannot enter the vent line when the cartridge is spinning or oscillating;
(b) a second vent line which is connected to and located radially inward from the lysis chamber such that the specimen within the lysis chamber cannot enter the vent line when the cartridge is spinning or oscillating;
(c) a third vent line which is connected to and located radially inward from the neutralization region such that the specimen within the neutralization region cannot enter the vent line when the cartridge is spinning or oscillating; and
(d) a fourth vent line which is connected to and located radially inward from the detection/hybridization chamber such that the specimen within the detection/hybridization chamber cannot enter the vent line when the cartridge is spinning or oscillating;
wherein the first, second, third and fourth vent lines are in fluid communication with each other.

76. A method of performing bacterial identification and quantification (IDQ) on a specimen, using the system described in any one of claims 49 through 75, the method comprising the steps of:
(a) securing the microfluidic cartridge with respect to the driving apparatus;
(b) dispensing a precise volume of the specimen into the fluid inlet port;
(c) using the rotating motor of the driving apparatus to rotate the microfluidic cartridge at a predetermined speed to allow the specimen within the fluid inlet port to flow into the incubation chamber;
(d) using the actuator to open the valve between the incubation chamber and the lysis chamber;
(e) using the rotating motor of the driving apparatus to rotate the microfluidic cartridge at a predetermined speed to allow the specimen within the incubation chamber to flow into the lysis chamber;
(f) using the rotating motor of the driving apparatus to rotate the microfluidic cartridge at a predetermined speed and angle such that the array of permanent magnets on the driving apparatus interact with the magnetic lysis puck and a chemical lysing agent in the lysis chamber, generating sheer force to break open the cell walls of the microorganisms and chemically degrade the cells and their biological material in the specimen and creating a lysate;
(g) using the actuator to open the valve between the lysis chamber and the neutralization region;
(h) using the rotating motor of the driving apparatus to rotate the microfluidic cartridge at a predetermined speed to allow the specimen lysate within the lysis chamber to flow into the neutralization region;
(i) using the rotating motor of the driving apparatus to oscillate the microfluidic cartridge at a predetermined oscillation frequency and angle to allow the buffer solution and series of detector probes functionalized with fluorescent signaling molecules within the neutralization region to neutralize the specimen lysate, creating neutralized lysate;
(i) using the actuator to open the valve between the neutralization region and the detection/hybridization chamber;
(k) using the rotating motor of the driving apparatus to rotate the microfluidic cartridge at a predetermined speed to allow the neutralized lysate within the neutralization region to flow into the detection/hybridization chamber;
(l) using the rotating motor of the driving apparatus to oscillate the microfluidic cartridge at a predetermined oscillation frequency to allow the neutralized lysate to hybridize and bind with complementary capture probes within the detection/hybridization chamber, wherein the detector probes are bound to the surface of a solid surface insert;
(m) using the actuator to open the valve between the detection/hybridization chamber and the waste chamber;
(n) using the rotating motor of the driving apparatus to rotate the microfluidic cartridge at a predetermined speed to allow the biological fluid in the detection/hybridization chamber to flow from the detection/hybridization chamber into the waste chamber;
(o) using the actuator to open the valve between the wash chamber and the detection/hybridization chamber;
(p) using the rotating motor of the driving apparatus to rotate the microfluidic cartridge at a predetermined speed to allow the wash buffer from the wash chamber to flow to the detection/hybridization chamber;
(q) using the rotating motor of the driving apparatus to position the detection/hybridization chamber above the detection system in the driving apparatus;
(r) imaging the biological fluid sample using an imaging device; and
(s) using the computer configured with image processing software, to convert the outputting light intensity information from the detection system to determine the identity and density of bacteria within the clinical specimen.

77. The method of claim 76, wherein the detection system is a fluorescent detection system.

78. The method of claim 76 or 77, wherein the imaging device is a fluorescent microscope.

79. The method disclosed in any of claims 76 through 78, wherein the predetermined speed at step (c) is about 4000 RPM.

80. The method disclosed in any of claims 76 through 78, wherein the predetermined speed at step (e) is about 4000 RPM.

81. The method disclosed in any of claims 76 through 78, wherein the predetermined speed at stage (f) is about 200 RPM.

82. The method disclosed in any of claims 76 through 78, wherein the predetermined speed at step (h) is about 4000 RPM.

83. The method disclosed in any of claims 76 through 78, wherein the predetermined oscillation frequency at step (i) is either 2 or 4 Hz.

84. The method disclosed in any of claims 76 through 78, wherein the predetermined speed at step (k) is about 4000 RPM.

85. The method disclosed in any of claims 76 through 78, wherein the predetermined oscillation frequency at step (l) is either 2 or 4 Hz.

86. The method disclosed in any of claims 76 through 78, wherein the predetermined speed at step (n) is about 4000 RPM.

87. The method disclosed in any of claims 76 through 78, wherein the predetermined speed at step (p) is about 2000 RPM.

88. A method of performing antimicrobial susceptibility testing (AST) on a specimen, using the system described in any one of claims 49 through 75, the method comprising the steps of:
(a) dispensing a predetermined amount of dried-down antibiotics into the incubation chamber of a microfluidic cartridge;
(b) securing the microfluidic cartridge with respect to the driving apparatus;
(c) dispensing a precise volume of the specimen into the fluid inlet port;
(d) using the rotating motor of the driving apparatus to rotate the microfluidic cartridge at a predetermined speed to allow the specimen within the fluid inlet port to flow into the incubation chamber;
(e) using the rotating motor of the driving apparatus to oscillate the microfluidic cartridge at a predetermined oscillation frequency and angle to allow turbulent mixing within the incubation chamber;
(f) incubating the incubation chamber;
(g) using the actuator to open the valve between the incubation chamber and the lysis chamber;
(h) using the rotating motor of the driving apparatus to rotate the microfluidic cartridge at a predetermined speed to allow the specimen within the incubation chamber to flow into the lysis chamber;
(i) using the rotating motor of the driving apparatus to rotate the microfluidic cartridge at a predetermined speed and angle such that the array of permanent magnets on the driving apparatus interact with the magnetic lysis puck and the chemical lysing agent in the lysis chamber, generating sheer force to break open the cell walls of microorganisms and chemically degrade the cells and the biological material contained in the specimen and creating a lysate;
(i) using the actuator to open the valve between the lysis chamber and the neutralization region;
(k) using the rotating motor of the driving apparatus to rotate the microfluidic cartridge at a predetermined speed to allow the specimen lysate within the lysis chamber to flow into the neutralization region;
(l) using the rotating motor of the driving apparatus to oscillate the microfluidic cartridge at a predetermined oscillation frequency and angle to allow the buffer solution and series of detector probes functionalized with fluorescent signaling molecules within the neutralization region to neutralize the specimen lysate, creating neutralized lysate;
(m) using the actuator to open the valve between the neutralization region and the detection/hybridization chamber;
(n) using the rotating motor of the driving apparatus to rotate the microfluidic cartridge at a predetermined speed to allow the neutralized lysate within the neutralization region to flow into the detection/hybridization chamber;
(o) using the rotating motor of the driving apparatus to the microfluidic cartridge at a predetermined oscillation frequency in order to allow the neutralized lysate to hybridize and bind with complementary capture probes within the detection/hybridization chamber, wherein the capture probes are bound to the surface of a solid surface insert;
(p) using the actuator to open the valve between the detection/hybridization chamber and the waste chamber;
(q) using the rotating motor of the driving apparatus to rotate the microfluidic cartridge at a predetermined speed to allow the biological fluid in the detection/hybridization chamber to flow from the detection/hybridization chamber into the waste chamber;
(r) using the actuator to open the valve between the wash chamber and the detection/hybridization chamber;
(s) using the rotating motor of the driving apparatus to rotate the microfluidic cartridge at a predetermined speed to allow the wash buffer from the wash chamber to flow to the detection/hybridization chamber;
(t) using the rotating motor of the driving apparatus to position the detection/hybridization chamber above the fluorescent detection system in the driving apparatus;
(u) imaging the biological fluid sample using a fluorescent microscope;
(v) using the computer configured with image processing software, to convert the outputting light intensity information from the fluorescent detection system to determine the density of bacteria within the specimen; and
(w) using the computer configured with image processing software to test the antibiotic susceptibility of the specimen.

89. The method of claim 88, wherein the detection system is a fluorescent detection system.

90. The method of claim 88 or 89, wherein the imaging device is a fluorescent microscope.

91. The method disclosed in any one of claims 88-90, wherein the time required to incubate the clinical specimen is around about 90 minutes.

92. The method disclosed in any one of claims 88-90, wherein the incubation step of step (f) is carried out at a temperature in the range of about range of about 25° C. to 45° C.

93. The method disclosed in any one of claims 88-90, wherein the incubation step of step (f) is carried out at a temperature of around about 37° C.

94. The method disclosed in any one of claims 88-90, wherein the predetermined speed at step (d) is about 4000 RPM.

95. The method disclosed in any one of claims 88-90, wherein the predetermined oscillation frequency at step (e) is between about 2 and 4 Hz.

96. The method disclosed in any one of claims 88-90, wherein the predetermined speed at step (h) is about 4000 RPM.

97. The method disclosed in any one of claims 88-90, wherein the predetermined speed at stage (i) is about 200 RPM.

98. The method disclosed in any one of claims 88-90, wherein the predetermined speed at step (k) is about 4000 RPM.

99. The method disclosed in any one of claims 88-90, wherein the predetermined oscillation frequency at step (1) is between about 2 and 4 Hz.

100. The method disclosed in any one of claims 88-90, wherein the predetermined speed at step (n) is about 4000 RPM.

101. The method disclosed in any one of claims 88-90, wherein the predetermined oscillation frequency at step (o) is between about 2 and 4 Hz.

102. The method disclosed in any one of claims 88-90, wherein the predetermined speed at step (q) is about 4000 RPM.

103. The method disclosed in any one of claims 88-90, wherein the predetermined speed at step (s) is about 2000 RPM.

104. A microfluidic cartridge comprising:
a body having a first surface and an opposing second surface;
a microfluidic flow path extending through the body and including an inlet to receive a fluid sample, the microfluidic flow path comprising:
(a) a first fluid conduit downstream from the inlet;
(b) a second fluid conduit downstream from the first fluid conduit; and
(c) a first blocking member positioned in the flow path between the first fluid conduit and the second fluid conduit and fluidly isolating the second fluid conduit from the first fluid conduit, the first blocking member being spaced inwardly from the upper surface and the lower surface;
wherein the first blocking member is configured to be at least partially destroyable via an actuator that is external the body portion when the cartridge is in use, thereby establishing fluid communication between the first fluid conduit and the second fluid conduit and allowing the fluid sample to flow from the first fluid conduit to the second fluid conduit.

105. The microfluidic cartridge of claim 104, wherein the first blocking member is meltable by a laser without melting the adjacent portions of the body.

106. The microfluidic cartridge of claim 104 or 105, wherein the actuator comprises at least one of a laser and an ultrasound transducer.

107. The microfluidic cartridge of claims 104 through 106, wherein the actuator comprises a laser and a first transmission portion of the first surface is disposed between the first blocking member and the laser so when the apparatus is in use the laser beam passes through the first transmission portion before reaching the blocking member, and the first transmission portion of the first surface remains intact.

108. A rotatable microfluidic cartridge for processing and analyzing at least one specimen, comprising:
a body portion having a first surface and an opposing second surface;
a fluid inlet region configured to receive the specimen; and
at least a first microfluidic flow path extending downstream from the fluid inlet port through the body of the cartridge,
wherein the cartridge is configured to be secured with respect to a driving apparatus and configured to be rotated about a rotational axis when the driving apparatus is in operation, and wherein the first microfluidic flow path comprises:
(a) a series of two or more fluid conduits connected to one another through a series of microfluidic flow channels that are configured to allow the specimen to flow between the fluid conduits when the cartridge is rotated or oscillated; and
(b) a blocking member positioned in the microfluidic flow channel between each of the two or more fluid conduits fluidly isolating each of the fluid conduits from one another, each of the blocking members being spaced inwardly from the first surface and second surface of the body;
wherein each of the blocking members is configured to be at least partially destroyable via the actuator that is external to body portion when the cartridge is in use, thereby establishing fluid communication between the two or more fluid conduits at a predetermined time and allowing the specimen to flow from the first fluid conduit to the second fluid conduit and so on.

109. The apparatus of claim 108, wherein the two or more fluid conduits comprise:
(a) an incubation chamber;
(b) a lysis chamber;
(c) a neutralization region; and
(d) a detection/hybridization chamber;
wherein (a), (b), (c), and (d) are all connected through a series of microfluidic channels that are configured to allow the specimen to flow from (a) to (b) to (c) to (d) when the cartridge is rotated or oscillated.

110. The apparatus disclosed in claim 108 or 109, wherein the incubation chamber is located downstream from the fluid inlet region, and wherein the incubation chamber is configured to receive the specimen from the fluid inlet region when the cartridge is rotated.

111. The apparatus disclosed in any of claims 108 through 110, wherein the lysis chamber is connected to and located downstream from the incubation chamber, and wherein the lysis chamber is configured to receive the specimen from the incubation chamber when the cartridge is rotated.

112. The apparatus disclosed in any of claims 108 through 111, wherein the neutralization region connected to and located downstream from the lysis chamber, and wherein the neutralization region is configured to receive the specimen from the lysis chamber when the cartridge is rotated.

113. The apparatus disclosed in any of claims 108 through 112, wherein the detection/hybridization chamber connected to and located downstream from the neutralization region, and wherein the detection/hybridization chamber is configured to receive the specimen from the neutralization region when the cartridge is rotated.
